SEQUENCE LISTING (2 sequences)

SEQ ID NO: 1 - DNA, 20 bp, Artificial Sequence
  Description of Artificial Sequence Synthetic oligonucleotide
tccatgacgttcctgatgct

SEQ ID NO: 2 - DNA, 20 bp, Artificial Sequence
  Description of Artificial Sequence Synthetic oligonucleotide
gggggacgatcgtcgggggg

(19) United States
(12) Patent Application Publication
(10) Pub. No.: US 2009/0304632 A1
(43) Pub. Date: Dec. 10, 2009

(54) HETEROCYCLIC NF-kB INHIBITORS

(76) Inventors: Johann LEBAN, Planegg-Martinsried (DE); Markus Hans-Jurgen Seifert, Munich (DE); Sylvia Lemstra, Planegg-Martinsried (DE)

Correspondence Address: MILLEN, WHITE, ZELANO & BRANIGAN, P.C., 2200 CLARENDON BLVD., SUITE 1400, ARLINGTON, VA, 22201, US

(21) Appl. No.: 12/234,108

(22) Filed: Aug. 6, 2009

(30) Foreign Application Priority Data
Sep. 19, 2007 (EP) 07116791.0

(51) Int. Cl. A61K 38/21; C07D 401/14; A61K 31/454; C07D 495/02; A61K 31/519; A61P 31/12

(52) U.S. Cl. 424/85.7; 546/199; 514/322; 544/278; 514/260.1

(57) ABSTRACT

The present invention relates to compounds of the general formula (Ihb) or pharmaceutically acceptable salts thereof with an acid or a base or a stereoisomer thereof for the prevention and/or treatment of a disease associated with increased cytokine release,

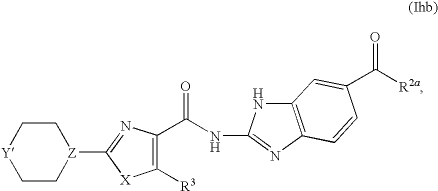

wherein
Y′ is O or NR^{2′};
Z is N or CR^{2′};
X is NR^{2′}, O or S;
R^{2′} is H, alkyl, —C(O)NR⁷, —C(O)R^{e}, cycloalkyl, haloalkyl, hydroxyalkyl, hydroxyalkylamino, alkylamino, heteroaryl, or aryl;
R³ is H, methyl, ethyl, methoxy, amine, alkylamine, morpholino, N-methylpiperazine, CF₃, or OCF₃;
R^{2a} is substituted or unsubstituted aryl, benzyl or heteroaryl.

The present invention relates to compounds of the general formula (Ihb) or a stereoisomer thereof or possible pharmaceutically acceptable salts thereof with an acid or a base, or pharmaceutically acceptable prodrugs of these compounds. The compounds of the invention can be used as a medicament and are exceptionally useful for the treatment of diseases associated with increased cytokine release in mammals, especially in humans. In particular, they are useful for the prevention and/or treatment of diseases that are caused by viral infections, such as infections with influenza viruses.

NF-κB (Nuclear Factor-κB) is a eukaryotic transcription factor of the rel family, which is located in the cytoplasm in an inactive complex, as a homo- or heterodimer. Predominantly it exists as a heterodimer composed of p50 and p65 subunits, bound to inhibitory proteins of the IκB family, usually IκB-α (D. Thanos et al., Cell 80, 529, 1995). NF-κB is activated in response to different stimuli, among which inflammatory cytokines, UV radiation, phorbol esters, bacterial and viral infections. Stimulation triggers the release of NF-κB from IκB in consequence of the phosphorylation and the following degradation of the IκB-α protein (P. A. Baeuerle et al., Annu. Rev. Immunol. 12, 141, 1995) by the proteasome. Once it is set free, NF-κB translocates in the nucleus where it binds to the DNA at specific κB-sites and induces the transcription of a variety of genes encoding proteins involved in controlling the immune and inflammatory responses, amongst others interleukins, TNF-α, the NO-synthase and the cyclooxygenase 2 (S. Grimm et al., J. Biochem. 290, 297, 1993). Accordingly, NF-κB is considered an early mediator of the immune and inflammatory responses and it is involved in the control of cell proliferation and in the pathogenesis of various human diseases, such as rheumatoid arthritis (H. Beker et al., Clin. Exp. Immunol. 99, 325, 1995), ischemia (A. Salminen et al., Biochem. Biophys. Res. Comm. 212, 939, 1995), arteriosclerosis (A. S. Baldwin, Annals Rev. Immunol. 212, 649, 1996), as well as in the pathogenesis of AIDS.

Inhibition of NF-κB mediated gene transcription can be accomplished through inhibition of phosphorylation of the inhibitory protein IκB, inhibition of IκB degradation, inhibition of NF-κB (p50/p65) nuclear translocation, the inhibition of NF-κB-DNA binding or NF-κB-mediated DNA transcription (J. C. Epinat et al., Oncogene 18, 6896, 1999).

Dysregulation of cytokine signaling is also an important characteristic of severe infectious diseases, e.g. septic shock or bird flu infections. Septic shock is triggered by an uncontrolled TNFα response to bacterial infection and bird flu infections are characterized by the so-called “cytokine-storm” both leading to multiple organ failures with a high mortality rate among patients. NF-κB has been shown recently to be involved in the propagation of several viruses, e.g. influenza A virus and HIV. Also an important role in propagation of hepatitis C is attributed to NF-κB.

Influenza infections pose an annually recurrent health problem with several millions of infected people during the winter months of the usual “flu season”. According to numbers published by the Robert-Koch-Institute, in the winter of 2002/3 16,000 to 20,000 people died from influenza infections in Germany alone. Presently, disease management largely relies on two strategies: vaccination programs and anti-viral drugs targeting virus surface or transmembrane proteins like the neuraminidase or the M2 ion channel. Vaccination programs suffer from several shortcomings like prediction of the correct virus subtype which will cause the next influenza epidemic, production costs, timeliness, supply, distribution and shelf-life of the vaccine. The emergence of a different virus strain may make the complete vaccination efforts obsolete. Treatment with anti-viral drugs on the other hand reduces the course of the disease usually only marginally by one or two days if taken in the early disease stages. Furthermore, the treatment may lead to the emergence of viral mutants, which can evade the antiviral effects of the drugs making them no responsive to the presently used antivirals. At present, the two most commonly used compounds for anti-influenza treatment are Oseltamivir (marketed under the brand name Tamiflu by Roche/Gilead) and Zaminivir (marketed under the brand name Relenza by GSK), both of which are neuraminidase inhibitors which block virus release from infected cells. Amantadine by Novartis, a M2 ion channel blocker inhibiting virus entry into cells, had previously been used in influenza therapy, but suffers from the fast spread of resistance mechanisms among viruses.

In the face of new emerging highly pathogenic and infective influenza virus strains, novel treatment options are urgently needed. Presently, influenza A subtype H5N1, colloquially called “bird flu”, represents a potentially pandemia causing influenza strain. The great influenza pandemics of the last century were initially highly pathogenic and infective bird flu viruses which adapted to the human host and became human transmittable. Although H5N1 up to now has only been sporadically transmitted from animals to humans, host adaptation and free distribution among humans can happen anytime due to the high mutation rate of influenza viruses and their genetic organization which is favoring gene swapping between different virus sub-types in co-infected hosts. Up to now 317 cases of H5N1 infections have been reported; 191 of these patients died (as of Jun. 29, 2007). Currently, no treatment option for patients suffering from such highly pathogenic viral infections is available. People infected with H5N1 suffer from fever, chill and symptoms comparable to a sepsis. In contrast to usual influenza infections which are cleared by the immune system after a couple of days, an infection with a highly pathogenic influenza virus like H5N1 causes the immune cells to release a high amount of cytokines, which results in a so-called cytokine storm, completely deregulating the immune system and causing death by multiple organ failure. In the absence of sufficient amounts of an effective vaccine and drugs active in late stage disease, a new approach to treat influenza infections is needed.

The present invention relates to compounds of the general formula (Ihb) or pharmaceutically acceptable salts thereof with an acid or a base, or pharmaceutically acceptable prodrugs or a stereoisomer thereof for the prevention and/or treatment of a disease associated with increased cytokine release.

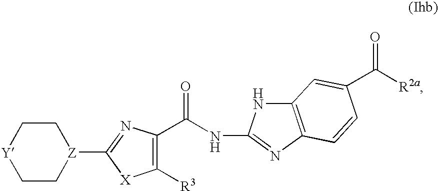

wherein
Y′ is O or NR^{2′};
Z is N or CR^{2′};
X is NR^{2′}, O or S;
R^{2′} is H, alkyl, —C(O)NR⁷, —C(O)R^{e}, cycloalkyl, haloalkyl, hydroxyalkyl, hydroxyalkylamino, alkylamino, heteroaryl, or aryl;
R³ is H, methyl, ethyl, methoxy, amine, alkylamine, morpholino, N-methylpiperazine, CF₃, or OCF₃and
R^{2a} is substituted or unsubstituted aryl, benzyl or heteroaryl.
R⁷, R^{7′}, R⁸ independently are H, halogen, alkyl, cycloalkyl, heterocycloalkyl, haloalkyl, hydroxyalkyl, hydroxyalkylamino, alkylamino, arylamino, heteroaryl, or aryl;
R^{e} independently reperesents H, —CN, —OH, —SH, —CO₂R^{4′}, —C(O)R^{4′}, —SO₂R^{4′}, —NR^{4′}R^{5′}, —C(O)NR⁷R⁸, —SO₂-alkyl, —SO₂R^{4′}, —SO₃R^{4′}, —N═CR^{4′}R^{5′}, —NR^{4′}C(O)R^{4″}, —NR^{4′}—CO-haloalkyl, —NO₂, —NR^{4′}—SO₂-haloalkyl, —NR^{4′}—SO₂-alkyl, —NR^{4′}—CO-alkyl, —NR^{4′}(CH₂)ₚheteroaryl, alkyl, hydroxyalkyl, cycloalkyl, alkylamino, aryl, hydroxyalkylamino, alkoxy, alkylthio, —O(CH₂)ₚ[O(CH₂)ₚ]_{q}OCH₃, —C(NR^{4″})NR^{4′}-benzimidazolyl, —C(NR^{4″})NR^{4′}-benzthiazolyl, —C(NR^{4″})NR^{4′}-benzoxazolyl, or heteroaryl;
R^{4′}, R^{4″}, R^{5′} independently are H, halogen, alkyl, —C(NR⁷)NR^{7′}R⁸, —(CH₂)ₚaryl, haloalkyl, —(CH₂)ₚNR⁷R⁸, —C(O)NR⁷R⁸, —N═CR⁷R⁸, —NR⁷C(O)R⁸, cycloalkyl, heterocycloalkyl, hydroxyalkyl, hydroxyalkylamino, alkylamino, heteroaryl, or aryl;
p=1-6
q=1-6

In formula (Ihb), the following substituents are preferred, alone or in combination:
Z is preferably CR^{2′}.
Y′ is preferably NR^{2′}.

In a preferred embodiment, the R^{2′} in the NR^{2′} of Y^{′} is preferably substituted or unsubstituted heteroaryl. In another preferred embodiment, the R^{2′} in the NR^{2′} of Y^{′} is aryl. In yet another preferred embodiment, the R^{2′} in the NR^{2′} of Y^{′} is benzyl. In a more preferred embodiment, the R^{2′} in the NR^{2′} of Y^{′} is pyrimidine or triazine. In another more preferred embodiment, the R^{2′} in the NR^{2′} of Y^{′} is a substituted or unsubstituted bicyclic heteroaryl, more preferably thienopyrimidine, quinazoline, purine, pyrazolopyrimidine or triazolpyrimidine, even more preferably thienopyrimidine.

In a preferred embodiment, X is S. In another preferred embodiment, X is O. In yet another preferred embodiment, X is NR^{2′}.
R³ is preferably H.
R^{2a} is preferably aryl or heteroaryl, more preferably phenyl.

The present invention also preferably relates to compounds of the general formula (Ihb-2) or pharmaceutically acceptable salts thereof with an acid or a base, or pharmaceutically acceptable prodrugs or a stereoisomer thereof for the prevention and/or treatment of a disease associated with increased cytokine release,

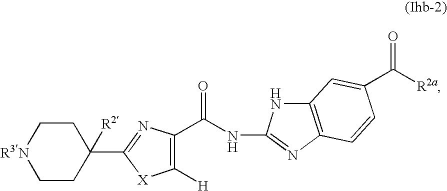

wherein
R^{3′} is substituted or unsubstituted heteroaryl or aryl.
X is NR^{2′}, O or S;
R^{2′} is H, alkyl, —C(O)NR⁷, —C(O)R^{e}, cycloalkyl, haloalkyl, hydroxyalkyl, hydroxyalkylamino, alkylamino, heteroaryl, or aryl;
R^{2a} is substituted or unsubstituted aryl or heteroaryl;
R⁷, R^{7′}, R⁸ independently are H, halogen, alkyl, cycloalkyl, heterocycloalkyl, haloalkyl, hydroxyalkyl, hydroxyalkylamino, alkylamino, arylamino, heteroaryl, or aryl;
R^{e} independently reperesents H, —CN, —OH, —SH, —CO₂R^{4′}, —C(O)R^{4′}, —SO₂R^{4′}, —NR^{4′}R^{5′}, —C(O)NR⁷R⁸, —SO₂-alkyl, —SO₂R^{4′}, —SO₃R^{4′}, —N═CR^{4′}R^{5′}, —NR^{4′}C(O)R^{4″}, —NR^{4′}—CO-haloalkyl, —NO₂, —NR^{4′}—SO₂-haloalkyl, —NR^{4′}—SO₂-alkyl, —NR^{4′}—CO-alkyl, —NR^{4′}(CH₂)ₚheteroaryl, alkyl, hydroxyalkyl, cycloalkyl, alkylamino, aryl, hydroxyalkylamino, alkoxy, alkylthio, —O(CH₂)ₚ[O(CH₂)ₚ]_{q}OCH₃, —C(NR^{4″})NR^{4′}-benzimidazolyl, —C(NR^{4″})NR^{4′}-benzthiazolyl, —C(NR^{4″})NR^{4′}-benzoxazolyl, or heteroaryl;
R^{4′}, R^{4″}, R^{5′} independently are H, halogen, alkyl, —C(NR⁷)NR^{7′} R⁸, —(CH₂)ₚaryl, haloalkyl, —(CH₂)ₚNR⁷R⁸, —C(O)NR⁷R⁸, —N═CR⁷R⁸, —NR⁷C(O)R⁸, cycloalkyl, heterocycloalkyl, hydroxyalkyl, hydroxyalkylamino, alkylamino, heteroaryl, or aryl.
p=1-6
q=1-6

The present invention also more preferably relates to compounds of the general formula (Ihb-3) or pharmaceutically acceptable salts thereof with an acid or a base, or pharmaceutically acceptable prodrugs or a stereoisomer thereof for the prevention and/or treatment of a disease associated with increased cytokine release,

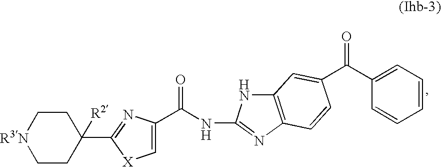

wherein
R^{3′} is a substituted or unsubstituted bicyclic heteroaryl;
X is NR^{2′}, O or S;
R^{2′} is H, alkyl, —C(O)NR⁷, —C(O)R^{e}, cycloalkyl, haloalkyl, hydroxyalkyl, hydroxyalkylamino, alkylamino, heteroaryl, or aryl;
R⁷, R^{7′}, R⁸ independently are H, halogen, alkyl, cycloalkyl, heterocycloalkyl, haloalkyl, hydroxyalkyl, hydroxyalkylamino, alkylamino, arylamino, heteroaryl, or aryl;
R^{e} independently reperesents H, —CN, —OH, —SH, —CO₂R^{4′}, —C(O)R^{4′}, —SO₂R^{4′}, —NR^{4′}R^{5′}, —C(O)NR⁷R⁸, —SO₂-alkyl, —SO₂R^{4′}, —SO₃R^{4′}, —N═CR^{4′}R^{5′}, —NR^{4′}C(O)R^{4″}, —NR^{4′}—CO-haloalkyl, —NO₂, —NR^{4′}—SO₂-haloalkyl, —NR^{4′}—SO₂-alkyl, —NR^{4′}—CO-alkyl, —NR^{4′}(CH₂)ₚheteroaryl, alkyl, hydroxyalkyl, cycloalkyl, alkylamino, aryl, hydroxyalkylamino, alkoxy, alkylthio, —O(CH₂)ₚ[O(CH₂)ₚ]_{q}OCH₃, —C(NR^{4″})NR^{4′}-benzimidazolyl, —C(NR^{4″})NR^{4′}-benzthiazolyl, —C(NR^{4″})NR^{4′}-benzoxazolyl, or heteroaryl;
R^{4′}, R^{4″}, R^{5′} independently are H, halogen, alkyl, —C(NR⁷)NR^{7′}R⁸, —(CH₂)ₚaryl, haloalkyl, —(CH₂)ₚNR⁷R⁸, —C(O)NR⁷R⁸, —N═CR⁷R⁸, —NR⁷C(O)R⁸, cycloalkyl, heterocycloalkyl, hydroxyalkyl, hydroxyalkylamino, alkylamino, heteroaryl, or aryl.
p=1-6
q=1-6

The present invention most preferably relates to compound (Ihb-4) or pharmaceutically acceptable salts thereof with an acid or a base, or pharmaceutically acceptable prodrugs or a stereoisomer thereof, for the prevention and/or treatment of a disease associated with increased cytokine release

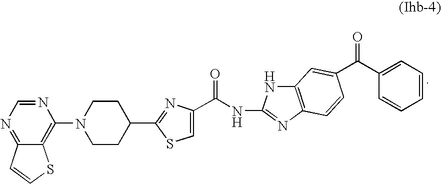

According to the present invention,
an alkyl group, if not stated otherwise, denotes a linear or branched C₁-C₆-alkyl, preferably a linear or branched chain of one to five carbon atoms, a linear or branched C₂-C₆-alkenyl or a linear or branched C₂-C₆-alkinyl group, which can optionally be substituted by one or more substituents R′;
the C₁-C₆-alkyl, C₂-C₆-alkenyl and C₂-C₆-alkinyl residue may be selected from the group comprising —CH₃, —C₂H₅, —CH═CH₂, —C≡CH, —C₃H₇, —CH(CH₃)₂, —CH₂—CH═CH₂, —C(CH₃)═CH₂, —CH═CH—CH₃, —C≡C—CH₃, —CH₂—C≡CH, —C₄H₉, —CH₂—CH(CH₃)₂, —CH(CH₃)—C₂H₅, —C(CH₃)₃, —C₅H₁₁, —C₆H₁₃, —C(R′)₃, —C₂(R′)₅, —CH₂—C(R′)₃, —C₃(R′)₇, —C₂H₄—C(R′)₃, —C₂H₄—CH═CH₂, —CH═CH—C₂H₅, —CH═C(CH₃)₂, —CH₂—CH═CH—CH₃, —CH═CH—CH═CH₂, —C₂H₄—C≡CH, —C≡C—C₂H₅, —CH₂—C≡C—CH₃, —C≡C—CH═CH₂, —CH═CH—C≡CH, —C≡C—C≡CH, —C₂H₄—CH(CH₃)₂, —CH(CH₃)—C₃H₇, —CH₂—CH(CH₃)—C₂H₅, —CH(CH₃)—CH(CH₃)₂, —C(CH₃)₂—C₂H₅, —CH₂—C(CH₃)₃, —C₃H₆—CH═CH₂, —CH═CH—C₃H₇, —C₂H₄—CH═CH—CH₃, —CH₂—CH═CH—C₂H₅, —CH₂—CH═CH—CH═CH₂, —CH═CH—CH═CH—CH₃, —CH═CH—CH₂—CH═CH₂, —C(CH₃)═CH—CH═CH₂, —CH═C(CH₃)—CH═CH₂, —CH═CH—C(CH₃)═CH₂, —CH₂—CH═C(CH₃)₂, C(CH₃)═C(CH₃)₂, —C₃H₆—C≡CH, —C≡C—C₃H₇, —C₂H₄—C≡C—CH₃, —CH₂—C≡C—C₂H₅, —CH₂—C≡C—CH═CH₂, —CH₂—CH═CH—C≡CH, —CH₂—C≡C—C≡CH, —C≡C—CH═CH—CH₃, —CH═CH—C≡C—CH₃, —C≡C—C≡C—CH₃, —C≡C—CH₂—CH═CH₂, —CH═CH—CH₂—C≡CH, —C≡C—CH₂—C≡CH, —C(CH₃)═CH—CH═CH₂, —CH═C(CH₃)—CH═CH₂, —CH═CH—C(CH₃)═CH₂, —C(CH₃)═CH—C≡CH, —CH═C(CH₃)—C≡CH, —C≡C—C(CH₃)═CH₂, —C₃H₆—CH(CH₃)₂, —C₂H₄—CH(CH₃)—C₂H₅, —CH(CH₃)—C₄H₉, —CH₂—CH(CH₃)—C₃H₇, —CH(CH₃)—CH₂—CH(CH₃)₂, —CH(CH₃)—CH(CH₃)—C₂H₅, —CH₂—CH(CH₃)—CH(CH₃)₂, —CH₂—C(CH₃)₂—C₂H₅, —C(CH₃)₂—C₃H₇, —C(CH₃)₂—CH(CH₃)₂, —C₂H₄—C(CH₃)₃, —CH(CH₃)—C(CH₃)₃, —C₄H₈—CH═CH₂, —CH═CH—C₄H₉, —C₃H₆—CH═CH—CH₃, —CH₂—CH═CH—C₃H₇, —C₂H₄—CH═CH—C₂H₅, —CH₂—C(CH₃)═C(CH₃)₂, —C₂H₄—CH═C(CH₃)₂, —C₄H₈—C—CH, —C≡C—C₄H₉, —C₃H₆—C≡C—CH₃, —CH₂—C≡C—C₃H₇, —C₂H₄—C≡C—C₂H₅;
R′ is independently H, —CO₂R″, —CONHR″, —CR″O, —SO₂NR″, —NR″—CO-haloalkyl, —NO₂, —NR″—SO₂-haloalkyl, —NR″—SO₂-alkyl, —SO₂-alkyl, —NR″—CO-alkyl, —CN, alkyl, cycloalkyl, alkylamino, alkoxy, —OH, —SH, alkylthio, hydroxyalkyl, hydroxyalkylamino, halogen, haloalkyl, haloalkyloxy, aryl, or heteroaryl;
R″ is independently H, haloalkyl, hydroxyalkyl, alkyl, cycloalkyl, aryl, or heteroaryl; a cycloalkyl group denotes a non-aromatic ring system containing three to eight carbon atoms, preferably four to eight carbon atoms, wherein one or more of the carbon atoms in the ring can be substituted by a group E, E being O, S, SO, SO₂, N, or NR″, R″ being as defined above; the C₃-C₈-cycloalkyl residue may be selected from the group comprising -cyclo-C₃H₅, -cyclo-C₄H₇, -cyclo-C₅H₉, —cyclo-C₆H₁₁, -cyclo-C₇H₁₃, -cyclo-C₈H₁₅, morpholine-4-yl, piperazinyl, 1-alkylpiperazine-4-yl;
an alkoxy group denotes an 0-alkyl group, the alkyl group being as defined above; the alkoxy group is preferably a methoxy, ethoxy, isopropoxy, t-butoxy or pentoxy group;
an alkylthio group denotes an S-alkyl group, the alkyl group being as defined above;
a haloalkyl group denotes an alkyl group which is substituted by one to five halogen atoms, the alkyl group being as defined above; the haloalkyl group is preferably a —C(R¹⁰)₃, —CR¹⁰(R^{10′})₂, —CR¹⁰(R^{10′})R^{10″}, —C₂(R¹⁰)₅, —CH₂—C(R¹⁰)₃, —CH₂—CR¹⁰(R^{10′})₂, —CH₂— CR¹⁰(R^{10′})R^{10″}, —C₃(R¹⁰)₇, or —C₂H₄—C(R¹⁰)₃, wherein R¹⁰ , R^{10′}, R^{10″} represent F, Cl, Br or I, preferably F;
a hydroxyalkyl group denotes an HO-alkyl group, the alkyl group being as defined above;
a haloalkyloxy group denotes an alkoxy group which is substituted by one to five halogen atoms, the alkyl group being as defined above; the haloalkyloxy group is preferably a —OC(R¹⁰)₃, —OCR¹⁰(R^{10′})₂, —OCR¹⁰(R^{10′})R^{10″}, —OC₂(R¹⁰)₅, —OCH₂—C(R¹⁰)₃, —OCH₂—CR¹⁰(R^{10′})₂, —OCH₂—CR¹⁰(R^{10′})R^{10″}, —OC₃(R¹⁰)₇ or —OC₂H₄—C(R¹⁰)₃, wherein R¹⁰, R^{10′}, R^{10″} represent F, Cl, Br or I, preferably F;
a hydroxyalkylamino group denotes an (HO-alkyl)₂-N— group or HO-alkyl-NH— group, the alkyl group being as defined above;
an alkylamino group denotes an HN-alkyl or N-dialkyl group, the alkyl group being as defined above;
a halogen group is chlorine, bromine, fluorine or iodine;
an aryl group denotes an aromatic group having five to fifteen carbon atoms, which can optionally be substituted by one or more substituents R′, where R′ is as defined above; the aryl group is preferably a benzyl group, a phenyl group, -o-C₆H₄—R′, -m-C₆H₄—R′, -p-C₆H₄—R′, 1-naphthyl, 2-naphthyl, 1-anthracenyl or 2-anthracenyl;
a heteroaryl group denotes a 5- or 6-membered heterocyclic group which contains at least one heteroatom selected from O, N, and S. This heterocyclic group can be fused to another aromatic ring. For example, this group can be selected from a thiadiazole, thiazol-2-yl, thiazol-4-yl, thiazol-5-yl, isothiazol-3-yl, isothiazol-4-yl, isothiazol-5-yl, oxazol-2-yl, oxazol-4-yl, oxazol-5-yl, isooxazol-3-yl, isooxazol-4-yl, isooxazol-5-yl, benzooxazol-2-yl, benzooxazol-4-yl, benzooxazol-5-yl, benzoisooxazol-3-yl, benzoisooxazol-4-yl, benzoisooxazol-5-yl, 1,2,4-oxadiazol-3-yl, 1,2,4-oxadiazol-5-yl, 1,2,5-oxadiazol-3-yl, 1,2,5-oxadiazol-4-yl, 1,2,4-thiadiazol-3-yl, 1,2,4-thiadiazol-5-yl, isothiazol-3-yl, isothiazol-4-yl, isothiazol-5-yl, benzoisothiazol-3-yl, benzoisothiazol-4-yl, benzoisothiazol-5-yl, 1,2,5-thiadiazol-3-yl, 1-imidazolyl, 2-imidazolyl, 1,2,5-thiadiazol-4-yl, 4-imidazolyl, benzoimidazol-4-yl, 1-pyrrolyl, 2-pyrrolyl, 3-pyrrolyl, 2-furanyl, 3-furanyl, 2-thienyl, 3-thienyl, 2-pyridyl, 3-pyridyl, 4-pyridyl, 2-pyranyl, 3-pyranyl, 4-pyranyl, 2-pyrimidinyl, 4-pyrimidinyl, 5-pyrimidinyl, 6-pyrimidinyl, 2,4-dimethoxy-6-pyrimidinyl, 3-pyridazinyl, 4-pyridazinyl, 2-pyrazinyl, 1-pyrazolyl, 3-pyrazolyl, 4-pyrazolyl, 1,2,3-triazol-4-yl, 1,2,3-triazol-5-yl, 1,2,4-triazol-3-yl, 1,2,4-triazol-5-yl, 1,3,5-triazol-6-yl, 2,4-dimethoxy-1,3,5-triazol-6-yl, 1H-tetrazol-2-yl, 1H-tetrazol-3-yl, tetrazolyl, acridyl, furazane, indazolyl, phenazinyl, carbazolyl, phenoxazinyl, indolizine, 2-indolyl, 3-indolyl, 4-indolyl, 5-indolyl, 6-indolyl, 7-indolyl, 1-isoindolyl, 3-isoindolyl, 4-isoindolyl, 5-isoindolyl, 6-isoindolyl, 7-isoindolyl, 2-indolinyl, 3-indolinyl, 4-indolinyl, 5-indolinyl, 6-indolinyl, 7-indolinyl, benzo[b]furanyl, benzofurazane, benzothiofurazane, benzotriazol-1-yl, benzotriazol-4-yl, benzotriazol-5-yl, benzotriazol-6-yl, benzotriazol-7-yl, benzotriazine, benzo[b]thiophenyl, benzimidazol-2-yl, 1H-benzimidazolyl, benzimidazol-4-yl, benzimidazol-5-yl, benzimidazol-6-yl, benzimidazol-7-yl, benzothiazolyl, quinazolinyl, quinoxazolinyl, cinnoline, quinolinyl, tetrahydroquinolinyl, isoquinolinyl, tetrahydroisoquinolinyl, purine, phthalazine, pteridine, thiatetraazaindene, thiatriazaindene, isothiazolopyrazine, isothiazolopyrimidine, pyrazolotriazine, pyrazolopyrimidine, tetrahydrothieno[3,4-d]imidazol-2-one, pyrazolo [5,1-c][1,2,4]triazine, 2,3 -dihydrobenzo [1,4]-dioxin-2-yl, 2,3-dihydrobenzo[1,4]-dioxin-3-yl, 2,3-dihydrobenzo[1,4]-dioxin-5-yl, 2,3-dihydrobenzo[1,4]-dioxin-6-yl, 2,6-dimethoxypyrimidin-3-yl, 2,6-dimethoxypyrimidin-4-yl, imidazopyridazine, imidazopyrimidine, imidazopyridine, imidazolotriazine, triazolotriazine, triazolopyridine, triazolopyrazine, triazolopyrimidine, or 4-[1,2,4]triazolo[4,3-a]pyridin-3-yl, 1-furo[2,3-c]pyridin-4-yl, 1-furo[2,3-c]pyridin-5-yl, 1-furo[2,3-c]pyridin-3-yl, and triazolopyridazine group. This heterocyclic group can be substituted by one or more substituents R′, wherein R′ is as defined above.

The compounds of formula (Ihb) may be obtained via various methods.

Piperidin-4yl-thiazole-4-carboxamide can be prepared by various methods described in the literature. One such example is the oxidation of the appropriate 2,5-dihydrothiazoles as described in Houben-Weyl, 2002, 730. The dihydrothiazoles can also be synthesized by methods described in the same reference or described in You, S., Razavi, H., Kelly, J. W. Angew. Chem. 2003, 115, 87 or Katritzky, A R., Cai, C., Suzuki, K., Singh, S K. J. Org. Chem. 2004, 69, 811-814 and references in both papers. Alternative methods were described by Yasuchika, S. et. al. Heterocycles, Vol. 57, No. 5, 2002.

Compounds of the present invention carrying a piperidin-4-yl substituent in the 2-position of the thiazole ring can, for example, be prepared as shown in the following scheme. This synthetic route is partially described in WO 2004/058750.

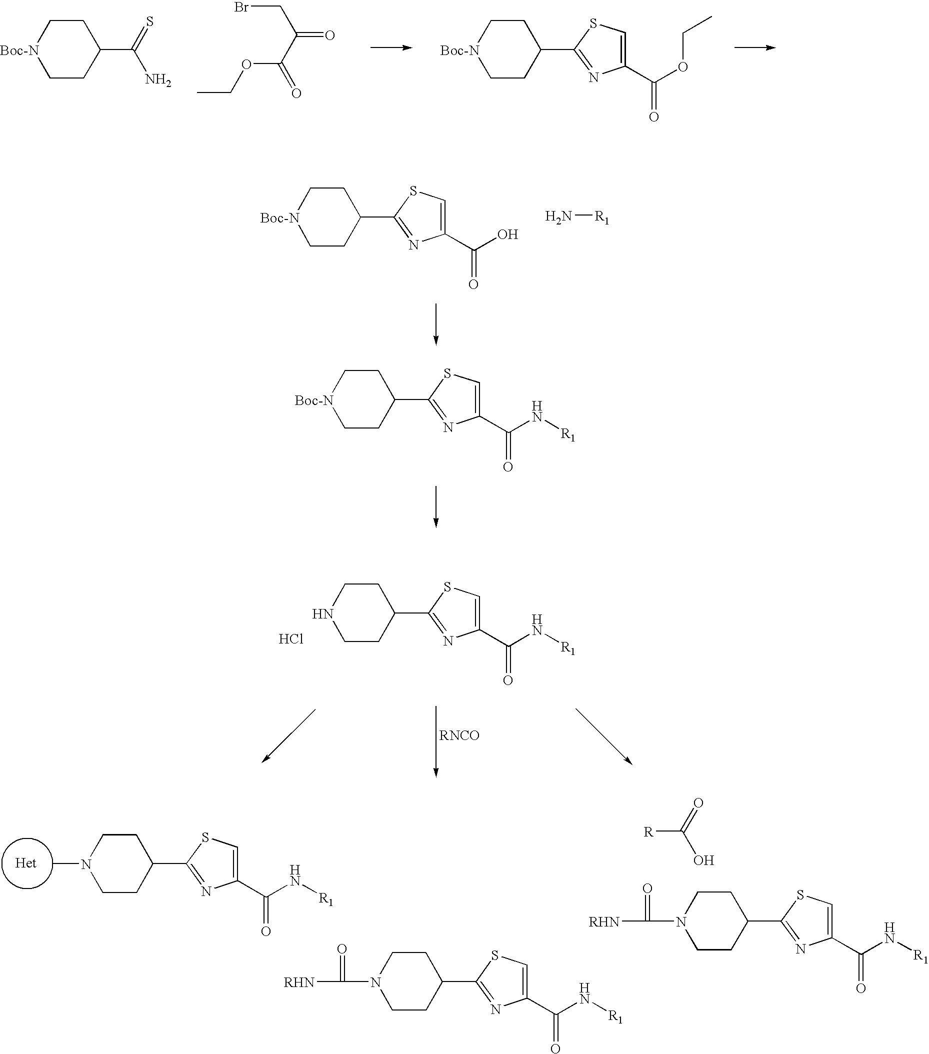

2-(1-(tert-butoxycarbonyl)piperidin-4-yl)thiazole-4-carboxylic acid can be converted to the appropriate R¹ amide by coupling with HBTU, DIPEA in DMF. The different R¹-amines are either commercially available or can be readily synthesized. The Boc-protection group can be removed under standard conditions, such as treatment with TFA for 2 to 3 hours at 0° or with 4 N HCl in dioxane for 2 to 3 hours. The deprotected piperidinoderivative HCl salt can then be converted to the corresponding amides, ureas and N-heterocyclic analogs as follows.

Urea substituted piperidino compounds can be synthesized by coupling with commercially available isocyanates in the presence of DIPEA. Piperidino compounds substituted with heterocycles can be synthesized by standard procedures, such as coupling with the corresponding chloroheterocycle in the presence of a base. Alternatively, piperidino compounds substituted with heterocycles can be obtained by palladium-mediated cross coupling. As a further alternative, hydroxypyridine derivatives can be coupled to the piperidino compound by the HBTU coupling method.

An alternative route to compounds of the present invention carrying a piperidin-4-yl substituent in the 2-position of the thiazole ring is shown in the following scheme.

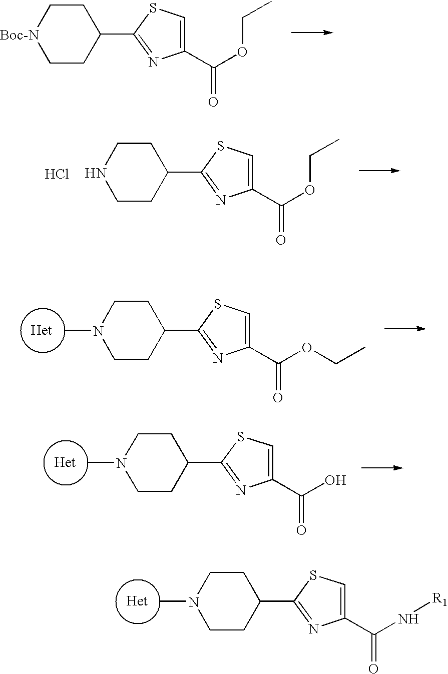

The Boc-protection group can be removed under standard conditions, such as treatment with TFA for 2 to 3 hours at 0° or with 4 N HCl in dioxane for 2 to 3 hours. The deprotected piperidinoderivative HCl salt can then be converted to the corresponding N-heterocyclic analog by various methods as described as described above.

For the compounds of the formula (Ihb) above, the term “stereoisomer” means cis/trans or E/Z isomerism. More particularly, the possible double bond(s) present in the various substituents of the compounds of the present invention can be E or Z configuration. These pure or impure geometrical isomers, alone or as a mixture, form an integral part of the compounds of the formula (Ihb). The term “stereoisomer” includes also all the isomeric forms, alone or as mixture, resulting from the presence of one or more axes and/or centers of symmetry in the molecules, and resulting in the rotation of a beam of polarized light. More particularly, it includes enantiomers and diastereomers, in pure form or as a mixture.

The compounds of the formula (Ihb) to be used according to the invention can form salts with inorganic or organic acids or bases. Examples of pharmaceutically acceptable salts comprise without limitation non-toxic inorganic or organic salts such as acetate derived from acetic acid, aconitate derived from aconitic acid, ascorbate derived from ascorbic acid, benzoate derived from benzoic acid, cinnamate derived from cinnamic acid, citrate derived from citric acid, embonate derived from embonic acid, enantate derived from heptanoic acid, formiate derived from formic acid, fumarate derived from fumaric acid, glutamate derived from glutamic acid, glycolate derived from glycolic acid, chloride derived from hydrochloric acid, bromide derived from hydrobromic acid, lactate derived from lactic acid, maleate derived from maleic acid, malonate derived from malonic acid, mandelate derived from mandelic acid, methanesulfonate derived from methanesulfonic acid, naphtaline-2-sulfonate derived from naphtaline-2-sulfonic acid, nitrate derived from nitric acid, perchlorate derived from perchloric acid, phosphate derived from phosphoric acid, phthalate derived from phthalic acid, salicylate derived from salicylic acid, sorbate derived from sorbic acid, stearate derived from stearic acid, succinate derived from succinic acid, sulphate derived from sulphuric acid, tartrate derived from tartaric acid, toluene-p-sulfate derived from p-toluene-sulfonic acid and others. Such salts can be produced by methods known to someone of skill in the art and described in the prior art.

Other salts like oxalate derived from oxalic acid, which is not considered as pharmaceutically acceptable can be appropriate as intermediates for the production of compounds of the formula (Ihb) or a pharmaceutically acceptable salt thereof or pharmaceutically acceptable prodrugs, or a stereoisomer thereof.

The invention covers the pharmaceutically acceptable salts, as indicated above, but also salts allowing a suitable separation or crystallization of the compounds of the formula (Ihb), such as the salts obtained with chiral amines.

The compounds of the formula (Ihb) above also comprise the prodrugs of these compounds. The term “prodrug” as used herein refers to compounds which once administered to the patient are not pharmaceutically active themselves (‘prodrugs’) but which are chemically and/or biologically transformed into their pharmaceutical active form (compounds of formula (Ihb)) in vivo, i.e. in the subject to which the compound is administered. Prodrugs include, for example, compounds of the invention wherein hydroxy, amine or sulfhydryl groups are bonded to any group that, when administered to a patient, cleaves to form the hydroxy, amine or sulfhydryl groups. Thus, representative examples of prodrugs include, but are not limited to, acetate, formate and benzoate derivatives of alcohol, sulfhydryl and amine functional groups of the compounds of the present invention. Further, in the case of carboxylic acid (—COOH), esters may be employed, such as methyl esters, ethyl esters, double esters and the like. Esters may be active in their own right and/or be hydrolysable under in vivo conditions in the human body.

“Treatment” according to the present invention is intended to mean complete or partial healing of a disease, or alleviation of a disease or stop of progression of a given disease.

The compounds of the present invention can be used for the prevention and/or treatment of infectious diseases caused by viruses, including opportunistic infections in a mammal, including a human. Said method comprises administering to the mammal an amount of at least one compound of the general formula (Ihb). The compounds according to the invention and medicaments prepared therewith are particularly useful for the treatment of diseases caused by influenza virus infections, more particular by influenza A virus infections.

After an influenza infection, cells switch on the NF-κB signaling pathway as a defense mechanism in order to clear the body from the invading pathogens. Influenza viruses, like a number of other viruses, in adaptation to the human environment are using this pathway for propagation in order to evade the immune system.

It has unexpectedly been found that compounds according to the present invention can be used in the treatment of viral infections, particularly infections with influenza A virus subtpes, such as, H1N1, H5N1, H2N2, or H3N2, by inhibiting NF-κB. An intervention in this cellular pathway will have at least two positive effects on the course of the disease. An inhibition of the NF-κB signaling pathway should impede further replication of viruses, restrict them to the infected cells and stop the spreading of the disease within the body of the treated persons, as well as transmission of the disease to other people by preventing the virus from leaving the cells. Moreover, inhibition of cytokine release should ameliorate the condition of infected people by reducing the deleterious effects of an overactive immune system and by blocking the ‘cytokine storm’. ‘Cytokine storm’ in this context is characterized as increased cytokine release which deregulates almost completely the immune system. Increased cytokine release encompasses increased cytokine release with and without deregulation of the immune system, wherein increased cytokine release with deregulation of the immune system is also called cytokine storm. A third aspect is the generation of resistances due to the usage of anti-viral medications. Presently available anti-influenza drugs like Amantadin, Tamiflu and Relenza target virus encoded proteins. Due to the high mutation rate of the viral genome, a selection pressure created by these drugs leads to a fast emergence of new, better-adapted and drug-resistant virus clades. Targeting a host protein or pathway indispensable for virus propagation and therefore hijacked by the invading organism, is a new, unprecedented approach, which evades a direct selection pressure on the virus. For a drug, which targets NF-κB, resistance to the drug is expected not to occur, since the drug target is of cellular origin instead of being a viral factor. Thus, the virus will not be able to adapt to the new medication by simple mutation or gene swapping, resulting in prevention of resistance formation. Thus, compounds according to the present invention should not favor the emergence of drug-resistant virus variants. Additionally, it will be possible to combine this treatment with the application of selected cytokines, especially interferons for which an inhibitory effect on influenza virus replication has already been shown. It will become possible to down-regulate overproduction of cytokines and at the same time give selected cytokines, which show an ameliorating effect on the course of the disease.

Thus, in one embodiment, the invention relates to the use of the compounds of the formula (Ihb) or a pharmaceutically acceptable salt or pharmaceutically acceptable prodrugs, or a stereoisomer thereof if desired with appropriate adjuvants and additives for use in a medicament for the prevention and/or treatment of diseases associated with increased cytokine release in mammals, especially in humans.

Diseases associated with dysregulated cytokine release are for example graft versus host disease (GVHD), adult respiratory distress syndrome (ARDS), sepsis and toxic shock syndrome, smallpox, systemic inflammatory response syndrome (SIRS), severe acute respiratory syndrome (SARS), Anthrax infections, dengue haemorrhagic fever/dengue shock syndrome, asthma, and hay fever, as well as type II diabetes and metabolic syndrome, in which release of cytokines is also dysregulated.

Thus, compounds of the present invention are used for the prevention and/or treatment of the above-mentioned diseases.

In a preferred embodiment, the invention relates to the use of the compounds of the formula (Ihb) or a pharmaceutically acceptable salt or pharmaceutically acceptable prodrugs, or a stereoisomer thereof if desired with appropriate adjuvants and additives for use in medicament for the prevention and/or treatment of diseases that are caused by viral infections.

The compounds of the present invention are especially suitable for use in the prevention and/or treatment of diseases related to an influenza virus infection.

Accordingly, in a more preferred embodiment, the invention relates to the use of the compounds of the formula (Ihb) or a pharmaceutically acceptable salt or pharmaceutically acceptable prodrugs, or a stereoisomer thereof if desired with appropriate adjuvants and additives for use in medicament for the prevention and/or treatment of diseases that are caused by infections with influenza viruses, especially influenza A virus infections.

In another even more preferred embodiment, the invention relates to the use of compounds of the formula (Ihb-2) or pharmaceutically acceptable salts or pharmaceutically acceptable prodrugs, or a stereoisomer thereof if desired with appropriate adjuvants and additives for use in a medicament for the prevention and/or treatment of diseases related to an influenza virus infection, especially those influenza virus infections which are associated with increased cytokine release. The compounds of the formula (Ihb-2) are especially useful for the prevention and/or treatment of diseases related to an influenza virus infection which are associated with a cytokine storm.

In another even more preferred embodiment, the invention relates to the use of compounds of the formula (Ihb-3) or pharmaceutically acceptable salts or pharmaceutically acceptable prodrugs, or a stereoisomer thereof if desired with appropriate adjuvants and additives for use in a medicament for the prevention and/or treatment of diseases related to an influenza virus infection, especially those influenza virus infections which are associated with increased cytokine release. The compounds of the formula (Ihb-3) are especially useful for the prevention and/or treatment of diseases related to an influenza virus infection which are associated with a cytokine storm.

In another even more preferred embodiment, the invention relates to the use of the compound of the formula (Ihb-4) or a pharmaceutically acceptable salt or pharmaceutically acceptable prodrug, or a stereoisomer thereof if desired with appropriate adjuvants and additives for use in a medicament for the prevention and/or treatment of diseases related to an influenza virus infection, especially those influenza virus infections which are associated with increased cytokine release. The compounds of the formula (Ihb-4) are especially useful for the prevention and/or treatment of diseases related to an influenza virus infection which are associated with a cytokine storm.

In an even more preferred embodiment, the invention relates to the use of compounds of the formula (Ihb) or pharmaceutically acceptable salts or pharmaceutically acceptable prodrugs, or a stereoisomer thereof if desired with appropriate adjuvants and additives for use in a medicament for the prevention and/or treatment of bird flu.

In an even more preferred embodiment, the invention relates to the use of compounds of the formula (Ihb-2) or pharmaceutically acceptable salts or pharmaceutically acceptable prodrugs, or a stereoisomer thereof if desired with appropriate adjuvants and additives for use in a medicament for the prevention and/or treatment of bird flu.

In an even more preferred embodiment, the invention relates to the use of compounds of the formula (Ihb-3) or pharmaceutically acceptable salts or pharmaceutically acceptable prodrugs, or a stereoisomer thereof if desired with appropriate adjuvants and additives for use in a medicament for the prevention and/or treatment of bird flu.

In an even more preferred embodiment, the invention relates to the use of the compound according to formula (Ihb-4) or a pharmaceutically acceptable salt or pharmaceutically acceptable prodrug, or a stereoisomer thereof if desired with appropriate adjuvants and additives for use in a medicament for the prevention and/or treatment of bird flu.

The compounds of the present invention are exceptionally suitable for use in the prevention and/or treatment of diseases related to an infection with one or more of the influenza subtypes H1N1, H5N1, H3N2 and H2N2.

In another even more preferred embodiment, the invention relates to the use of compounds of the formula (Ihb) or pharmaceutically acceptable salts or pharmaceutically acceptable prodrugs, or a stereoisomer thereof if desired with appropriate adjuvants and additives for use in a medicament for the prevention and/or treatment of bird flu, as for example caused by influenza virus subtypes H1N1, H5N1, H3N2 and H2N2.

In another even more preferred embodiment, the invention relates to the use of compounds of the formula (Ihb-2) or pharmaceutically acceptable salts or pharmaceutically acceptable prodrugs, or a stereoisomer thereof if desired with appropriate adjuvants and additives for use in a medicament for the prevention and/or treatment of bird flu, as for example caused by influenza virus subtypes H1N1, H5N1, H3N2 and H2N2.

In another even more preferred embodiment, the invention relates to the use of compounds of the formula (Ihb-3) or pharmaceutically acceptable salts or pharmaceutically acceptable prodrugs, or a stereoisomer thereof if desired with appropriate adjuvants and additives for use in a medicament for the prevention and/or treatment of bird flu, as for example caused by influenza virus subtypes H1N1, H5N1, H3N2 and H2N2.

In another even more preferred embodiment, the invention relates to the use of the compound of the formula (Ihb-4) or a pharmaceutically acceptable salt or pharmaceutically acceptable prodrug, or a stereoisomer thereof if desired with appropriate adjuvants and additives for use in a medicament for the prevention and/or treatment of bird flu, as for example caused by influenza virus subtypes H1N1, H5N1, H3N2 and H2N2.

The inventive compounds, especially a specific compound, Ihb-4, is 1) a very potent NF-κB reporter gene assay inhibitor (IC₅₀ in 100 nm range); 2) not cytotoxic; 3) inhibits human lymphocyte and mouse T-cell proliferation; 4) actually inhibits cytokine release; 5) inhibits influenza virus replication while maintaining host cell viability; does not induce viral resistance; and has synergistic antiviral effects when used in combination with INF-α; 6) displays in vivo efficacy in influenza mouse models 7) and shows activity versus a number of human cancer cell lines, especially multiple myeloma;

Bird Flu, as mentioned herein, specifies viral infections caused by influenza A virus subtypes, in particular influenza A subtypes H5N1, H1N1 (Spanish flu), H2N2 (Asian Flu) and H3N2 (Hong Kong Flu). All of the afore-mentioned influenza types are associated with increased cytokine release. Subject of the present invention is the use of compounds of formula (Ihb) or a pharmaceutically acceptable salt or pharmaceutically acceptable prodrugs, or a stereoisomer thereof in influenza types which cause an increased cytokine release in the body of the infected persons

Furthermore, the invention relates to a method of treatment or prevention of diseases, which comprises the administration of an effective amount of compounds of the formula (Ihb) or a pharmaceutically acceptable salt or pharmaceutically acceptable prodrugs, or a stereoisomer thereof.

The invention also provides a pharmaceutical composition comprising a compound of formula (Ihb), in free form or in the form of pharmaceutically acceptable salts and pharmaceutically acceptable prodrugs, together with a pharmaceutically acceptable diluent or carrier therefore.

The compounds of formula (Ihb) and their pharmacologically acceptable salts can be administered to animals, preferably to mammals, and in particular to humans as therapeutics per se, as mixtures with one another or in the form of pharmaceutical preparations which allow enteral or parenteral use and which as active constituent contain an effective dose of at least one compound of the formula (Ihb) or a salt thereof, in addition to customary pharmaceutically innocuous excipients and additives. The compounds of formula (Ihb) can also be administered in form of their salts, which are obtainable by reacting the respective compounds with physiologically acceptable acids and bases.

The production of medicaments containing the compounds of formula (Ihb) according to the invention and their application can be performed according to well-known pharmaceutical methods.

While the compounds of formula (Ihb) according to the invention for use in therapy may be administered in the form of the raw chemical compound, it is preferred to introduce the active ingredient, optionally in the form of a physiologically acceptable salt in a pharmaceutical composition together with one or more adjuvants, excipients, carriers, buffers, diluents, and/or other customary pharmaceutical auxiliaries. Such salts of the compounds may be anhydrous or solvated.

In a preferred embodiment, the invention provides medicaments comprising compounds of formula (Ihb) according to the invention, or a pharmaceutically acceptable salt or pharmaceutically acceptable prodrugs or a stereoisomer thereof, together with one or more pharmaceutically acceptable carriers thereof, and, optionally, other therapeutic and/or prophylactic ingredients. The carrier(s) must be “acceptable” in the sense of being compatible with the other ingredients of the formulation and not harmful to the recipient thereof.

A medicament of the invention may be those suitable for oral, rectal, bronchial, nasal, topical, buccal, sub-lingual, transdermal, vaginal or parenteral (including cutaneous, subcutaneous, intramuscular, intraperitoneal, intravenous, intraarterial, intracerebral, intraocular injection or infusion) administration, or those in a form suitable for administration by inhalation or insufflation, including powders and liquid aerosol administration, or by sustained release systems. Suitable examples of sustained release systems include semipermeable matrices of solid hydrophobic polymers containing the compound of the invention, which matrices may be in form of shaped articles, e.g. films or microcapsules.

The compounds according to the invention, together with a conventional adjuvant, carrier, or diluent, may thus be placed into the form of medicament and unit dosages thereof. Such forms include solids, and in particular tablets, filled capsules, powder and pellet forms, and liquids, in particular aqueous or non-aqueous solutions, suspensions, emulsions, elixirs, and capsules filled with the same, all for oral use, suppositories for rectal administration, and sterile injectable solutions for parenteral use. Such medicament and unit dosage forms thereof may comprise conventional ingredients in conventional proportions, with or without additional active compounds or principles, and such unit dosage forms may contain any suitable effective amount of the active ingredient commensurate with the intended daily dosage range to be employed.

The compound useable according to the invention can be administered in a wide variety of oral and parenteral dosage forms. It will be obvious to those skilled in the art that the following dosage forms may comprise, as the active component, either a compound of formula (Ihb) according to the invention or a pharmaceutically acceptable salt or stereoisomer thereof.

For preparing a medicament from a compound of formula (Ihb) pharmaceutically acceptable carriers can be either solid or liquid. Solid form preparations include powders, tablets, pills, capsules, cachets, suppositories, and dispersible granules. A solid carrier can be one or more substances, which may also act as diluents, flavoring agents, solubilizers, lubricants, suspending agents, binders, preservatives, tablet disintegrating agents, or an encapsulating material.

In powders, the carrier is a finely divided solid, which is in a mixture with the finely divided active component. In tablets, the active component is mixed with the carrier having the necessary binding capacity in suitable proportions and compacted in the shape and size desired. Suitable carriers are magnesium carbonate, magnesium stearate, talc, sugar, lactose, pectin, dextrin, starch, gelatin, tragacanth, methylcellulose, sodium carboxymethylcellulose, a low melting wax, cocoa butter, and the like. The term “preparation” is intended to include the formulation of the active compound with encapsulating material as carrier providing a capsule in which the active component, with or without carriers, is surrounded by a carrier, which is thus in association with it. Similarly, cachets and lozenges are included. Tablets, powders, capsules, pills, cachets, and lozenges can be used as solid forms suitable for oral administration.

For preparing suppositories, a low melting wax, such as a mixture of fatty acid glyceride or cocoa butter, is first melted and the active component is dispersed homogeneously therein, as by stirring. The molten homogenous mixture is then poured into convenient sized moulds, allowed to cool, and thereby to solidify. Compositions suitable for vaginal administration may be presented as pessaries, tampons, creams, gels, pastes, foams or sprays containing in addition to the active ingredient such carriers as are known in the art to be appropriate. Liquid preparations include solutions, suspensions, and emulsions, for example, water or water-propylene glycol solutions. For example, parenteral injection liquid preparations can be formulated as solutions in aqueous polyethylene glycol solution.

The compounds of formula (Ihb) according to the present invention may thus be formulated for parenteral administration (e.g. by injection, for example bolus injection or continuous infusion) and may be presented in unit dose form in ampoules, pre-filled syringes, small volume infusion or in multi-dose containers with an added preservative. The compositions may take such forms as suspensions, solutions, or emulsions in oily or aqueous vehicles, and may contain formulation agents such as suspending, stabilizing and/or dispersing agents. Alternatively, the active ingredient may be in powder form, obtained by aseptic isolation of sterile solid or by lyophilization from solution, for constitution with a suitable vehicle, e.g. sterile, pyrogen-free water, before use.

Aqueous solutions suitable for oral use can be prepared by dissolving the active component in water and adding suitable colorants, flavors, stabilizing and thickening agents, as desired. Aqueous suspensions suitable for oral use can be made by dispersing the finely divided active component in water with viscous material, such as natural or synthetic gums, resins, methylcellulose, sodium carboxymethylcellulose, or other well known suspending agents.

Also included are solid form preparations, which are intended to be converted, shortly before use, to liquid form preparations for oral administration. Such liquid forms include solutions, suspensions, and emulsions. These preparations may contain, in addition to the active component, colorants, flavors, stabilizers, buffers, artificial and natural sweeteners, dispersants, thickeners, solubilizing agents, and the like.

In one embodiment of the present invention, the medicament is applied topically or systemically or via a combination of the two routes.

In an especially preferred embodiment of the present invention the medicament is applied topically. This reduces possible side effects and limits the necessary treatment to those areas affected.

Preferably the medicament is prepared in form of an ointment, a gel, a plaster, an emulsion, a lotion, a foam, a cream of a mixed phase or amphiphilic emulsion system (oil/water-water/oil mixed phase), a liposome, a transfersome, a paste or a powder.

Ointments and creams may, for example, be formulated with an aqueous or oily base with the addition of suitable thickening and/or gelling agents. Lotions may be formulated with an aqueous or oily base and will in general also contain one or more emulsifying agents, stabilizing agents, dispersing agents, suspending agents, thickening agents, or coloring agents.

Compositions suitable for topical administration in the mouth include lozenges comprising the active agent in a flavored base, usually sucrose and acacia or tragacanth; pastilles comprising the active ingredient in an inert base such as gelatin and glycerin or sucrose and acacia; and mouthwashes comprising the active ingredient in a suitable liquid carrier.

Solutions or suspensions are applied directly to the nasal cavity by conventional means, for example with a dropper, pipette or spray. The compositions may be provided in single or multi-dose form. In the latter case of a dropper or pipette, this may be achieved by the patient administering an appropriate, predetermined volume of the solution or suspension. In the case of a spray, this may be achieved for example by means of a metering atomising spray pump.

Administration to the respiratory tract may also be achieved by means of an aerosol formulation in which the active ingredient is provided in a pressurised pack with a suitable propellant such as a chlorofluorocarbon (CFC) for example dichlorodifluoromethane, trichlorofluoromethane, or dichlorotetrafluoroethane, carbon dioxide, or other suitable gas. The aerosol may conveniently also contain a surfactant such as lecithin. The dose of drug may be controlled by provision of a metered valve.

Alternatively the active ingredients may be provided in the form of a dry powder, for example a powder mix of the compound in a suitable powder base such as lactose, starch, starch derivatives such as hydroxypropylmethyl cellulose and polyvinylpyrrolidone (PVP). Conveniently the powder carrier will form a gel in the nasal cavity. The powder composition may be presented in unit dose form for example in capsules or cartridges of, e.g., gelatin, or blister packs from which the powder may be administered by means of an inhaler.

In compositions intended for administration to the respiratory tract, including intranasal compositions, the compound will generally have a small particle size for example of the order of 5 microns or less. Such a particle size may be obtained by means known in the art, for example by micronization.

When desired, compositions adapted to give sustained release of the active ingredient may be employed.

The pharmaceutical preparations are preferably in unit dosage forms. In such form, the preparation is subdivided into unit doses containing appropriate quantities of the active component. The unit dosage form can be a packaged preparation, the package containing discrete quantities of preparation, such as packaged tablets, capsules, and powders in vials or ampoules. Also, the unit dosage form can be a capsule, tablet, cachet, or lozenge itself, or it can be the appropriate number of any of these in packaged form. Tablets or capsules for oral administration and liquids for intravenous administration and continuous infusion are preferred compositions.

Further details on techniques for formulation and administration may be found in the latest edition of Remington's Pharmaceutical Sciences (Maack Publishing Co. Easton, Pa.).

Pharmaceutical compositions can also contain two or more compounds of the formula (Ihb) or their pharmacologically acceptable salts and also other therapeutically active substances.

Thus, the compounds of the present invention can be used in the form of one compound alone or in combination with other active compounds—for example with medicaments already known for the treatment of the aforementioned diseases, whereby in the latter case a favorable additive, amplifying effect is noticed. Suitable amounts to be administered to humans may range from 5 to 500 mg.

Surprisingly, the combination of a compound of the present invention with interferon-α leads to strong synergistic anti-viral effects, while toxicity is antagonized by the combination of the two agents. Notably, combination of the compounds of the present invention, in particular the compound according to formula (Ihb-4) with INFα shows stronger synergism in anti-viral efficacy and toxicity antagonism than IFNα combination with ribavirin.

In another embodiment, the compounds of the present invention or a composition thereof can be used for the prevention and/or treatment of a disease associated with increased cytokine release, whereby the compound or composition is administered in combination with interferon alpha.

To prepare the pharmaceutical preparations, pharmaceutically inert inorganic or organic excipients can be used. To prepare pills, tablets, coated tablets and hard gelatin capsules, for example, lactose, corn starch or derivatives thereof, talc, stearic acid or its salts, etc. can be used. Excipients for soft gelatin capsules and suppositories are, for example, fats, waxes, semi-solid and liquid polyols, natural or hardened oils etc. Suitable excipients for the production of solutions and syrups are, for example, water, sucrose, invert sugar, glucose, polyols etc. Suitable excipients for the production of injection solutions are, for example, water, alcohols, glycerol, polyols or vegetable oils.

The dose can vary within wide limits and is to be suited to the individual conditions in each individual case. For the above uses the appropriate dosage will vary depending on the mode of administration, the particular condition to be treated and the effect desired. In general, however, satisfactory results are achieved at dosage rates of about 1 to 100 mg/kg animal body weight preferably 1 to 50 mg/kg. In general, suitable dosage rates for larger mammals, for example humans, may be of the order of from about 10 mg to 3 g/day, conveniently administered once, in divided doses 2 to 4 times a day, or in sustained release form.

In general, a daily dose of approximately 10 mg to 5000 mg, preferably 50 to 500 mg, per human individual is appropriate in the case of the oral administration. In the case of other administration forms too, the daily dose is in similar ranges. For topical delivery, depending on the permeability of the skin, the type and the severity of the disease and dependent on the type of formulation and frequency of application, different concentrations of active compounds within the medicament can be sufficient to elicit a therapeutic effect by topical application. Preferably the concentration of an active compound or a pharmaceutically acceptable salt thereof or a physiologically functional derivative or a stereoisomer thereof within a medicament according to the invention is in the range of between 1 μmol/l and 100 mmol/l.

The following examples and figures are included to demonstrate preferred embodiments of the invention. It should be appreciated by those of skill in the art that the techniques disclosed in the examples that follow represent techniques discovered by the inventors to function well in the practice of the invention, and thus can be considered preferred modes for its practice. However, those of skill in the art should, in light of the present disclosure, appreciate that many changes can be made in the specific embodiments that are disclosed without departing from the spirit and scope of the invention as set out in the appended claims. All references cited are incorporated herein by reference.

### EXAMPLES

Abbreviations: min, minute(s); h, hour(s); r.t., room temperature; t-, tert-.

NMR spectra: Bruker Avance 300 MHz. The spectra were recorded at 300 MHz (¹H-NMR), respectively, using the residual solvent peak as an internal standard (DMSO-d₆, δ_{H}=2.49; CD₃OD, δ_{H}=3.31; CDCl₃, δ_{H}=7.26; CD₃CN, δ_{H}=1.93; (CD₃)₂CO, δ_{H}=2.05).

Analytical LC/ESI-MS: 2× Waters 600 Multisolvent Delivery System. 50 μl sample loop. Column, Chromolith Speed ROD RP18e (Merck, Darmstadt), 50×4.6 mm, with 2 μm prefilter (Merck). Eluent A, H₂O+0.1% HCO₂H; eluent B, MeCN. Gradient, 5% B to 100% B within 5 min; flow, 3 ml/min. Waters LCZ single quadrupol mass spectrometer with electrospray source. MS method, MS8minPM-80-800-20V; positive/negative ion mode scanning, m/z 80-800 in 1 s; capillary, 3.5 kV; cone voltage, 20 V; multiplier voltage, 400 V; probe and desolvation gas temperature, 120° C. and 350° C., respectively. Waters 2487 Dual λ Absorbance Detector, set to 254 nm.

Preparative HPLC-MS: Waters 600 Multisolvent Delivery System with peparative pump heads. 2000 μl or 5000 μl sample loop. Column, Waters X-Terra RP18, 7 μm, 19×150 mm with X-Terra RP18 guard cartridge 7 μm, 19×10 mm; used at flow rate 20 ml/min or YMC ODS-A, 120 Å, 40×150 mm with X-Terra RP18 guard cartridge 7 μm, 19×10 mm; used at flow rate 50 ml/min. Make-up solvent: MeCN—H₂O—HCO₂H 80:20:0.05 (v:v:v). Eluent A, H₂O+0.1% HCO₂H; eluent B, MeCN. Different linear gradients from 5-100% eluent B, adapted to sample. Injection volume: 500 μl-2000 μl depending on sample. Waters ZQ single quadrupol mass spectrometer with electrospray source. Positive or negative ion mode scanning m/z 80-800 in 1 s; capillary, 3.5 kV or 3.0 kV; cone voltage, 20 V; multiplier voltage, 400 V; probe and desolvation gas temperature, 120° C. and 350° C., respectively. Waters Fraction Collector II with mass-triggered fraction collection. Waters 996 photo diode array detector.

Synthesis of 4-(Methoxy-methyl-carbamoyl)-piperidine-1-carboxylic acid t-butyl ester

Piperidine-1,4-dicarboxylic acid mono-tert-butyl ester (1.0 eq, 21.8 mmol) was dissolved under inert conditions in 35 ml dry N, N-dimethylformamide. O,N-dimethyl-hydroxylamine hydrochloride (1.03 eq, 22.5 mmol), benzotriazol-1-ol monohydrate (1.03 eq, 22.5 mmol) and triethylamine (1.5 eq, 32.7 mmol) were added. The reaction mixture was cooled to 0° C., N-(3-Dimethylaminopropyl)-N-ethylcarbodiimid hydrochloride (1.0 eq, 21.8 mmol) was added over a period of 10 minutes and the mixture was stirred vigorously at 0° C. for 1 h and at r.t. for 18 h.

The solvent was removed under vaccum and the residue was suspended in 400 ml ethylacetate. The organic layer was extracted 3 times with 100 ml of 1 M citric acid, aqueous sodium carbonate and twice with 100 ml brine, dried over MgSO₄ and filtered. The solvent was removed and the residue was purified by distillation resulting in a yield of 80%.

Synthesis of 4-Formyl-piperidine-1-carboxylic acid t-butyl ester

4-(methoxy-methyl-carbamoyl)-piperidine-1-carboxylic acid tert-butyl ester (1.0 eq, 16.4 mmol) was dissolved in 100 ml dry tetrahydrofurane under inert atmosphere. This solution was added dropwise over a period of 1 h to a suspension of lithiumalanate (3.0 eq, 49.6 mmol) in 70 ml dry tetrahydrofurane at −50° C. During the adding of the mixture, the temperature was held at −50° C. and then allowed to warm to 0° C. within 3 h.

The mixture was cooled to −78° C. and quenched carefully with 100 ml 1 M citric acid. The mixture was warmed up to r.t. and diluted with 400 ml ethylacetate. The phases were separated and the aqueous phase was extracted 3 times with 70 ml ethylacetate. The combined organic layers were extracted 3 times with 100 ml 1 M citric acid, aqueous sodium carbonate and 2 times with 100 ml brine, dried over MgSO₄ and filtrated. The solvent was removed and the residue was purified by distillation resulting in a yield of 85%

Synthesis of 4-(4-Ethoxycarbonyl-4,5-dihydro-thiazol-2-yl)-piperidine-1-carboxylic acid t-butyl ester

4-formyl-piperidine-1-carboxylic acid tert-butyl ester (1.0 eq, 13 mmol) was dissolved under inert conditions in 40 ml toluene. To this solution L-cystein ethylester hydrochloride (1.6 eq, 21 mmol) and triethylamine (1.6 eq, 21 mmol) were added. The mixture was refluxed for 14 h. The generated water was removed with a Dean & Stark trap.

The solvent was removed and the residue was dissolved in 100 ml ethylacetate. The organic layer was extracted 3 times with 50 ml 1 M citric acid, aqueous potassium hydrogen carbonate and 2 times with 50 ml brine, dried over MgSO₄ and filtrated. The solvent was removed and the residue was purified by silica gel chromatography using a PE/EA 4:1 gradient. Yield: 75%

Synthesis of 4-(4-Ethoxycarbonyl-thiazol-2-yl)-piperidine-1-carboxylic acid t-butyl ester

4-(4-Ethoxycarbonyl-4,5-dihydro-thiazol-2-yl)-piperidine-1-carboxylic acid tert-butyl ester (1.0 eq, 8.7 mmol) was solved in 160 ml toluene under inert conditions. To this solution MnO₂ (15.0 eq, 130 mmol) was added. The reaction was heated to 70° C. under stirring for 5 h. The mixture was filtered over celite and the filtration agent was washed 3 times with 30 ml toluene and ethylacetate. The combined organic layers were distilled in vacuo. The residue was purified by silica gel chromatography using a DCM/MeOH 95:5 gradient. Yield: 30%

C-terminal functionalisation

4-(4-Ethoxycarbonyl-thiazol-2-yl)-piperidine-1-carboxylic acid tert-butyl ester (1.0 eq, 2.9 mmol) was dissolved under inert gas in 40 ml dioxane. Under stirring 1.5 ml aqueous 2 N NaOH was added dropwise over a period of 10 min. Afterwards the mixture was stirred for 2 h at r.t.

The reaction was neutralized with 2 N HCl and the solvent was evaporated in vacuo. The residue was dissolved in 50 ml ethylacetate. The organic layer was extracted 3 times with 10 ml of 1 M citric acid and water, dried over MgSO₄ and filtered. The solvent was removed and the residue was dried in vacuo. Yield 95%

4-(4-Carboxy-thiazol-2-yl)-piperidine-1-carboxylic acid tert-butyl ester (1 eq) was dissolved under inert conditions in dry dimethylacetamide (0.03 mmol/ml). To this solution aryl- or alkylamine (1 eq), diisopropylethylamine (2 eq) and O-benzotriazol-1-yl-N,N,N′,N′-tetramethyluronium hexafluorophosphate (2 eq) was added. The reaction mixture was stirred for 12 h at r.t.

The solvent was removed in vacuo and the residue was dissolved in ethylacetate. The organic layer was extracted 3 times with 1 M citric acid, aqueous potassium hydrogen carbonate and 2 times with brine, dried over MgSO₄ and filtred. The solvent was removed and the residue was purified by silica gel chromatography using a DCM/MeOH 95:5 gradient. Yield: 40-80%

### N-terminal Functionalisation

The N-protected substrate was treated under inert condition with 4 M HCl/dioxane (conc. 0.03 mmol substrate in 1 mL HCl/dioxane) and was stirred for 2 h at r.t.

The solvent was removed in vacuo to yield the HCl salt of the free amine without further purification.

The free amino compound (1 eq) was dissolved under inert conditions in dry dimethylacetamide (0.03 mmol/ml). To this solution (1 eq) of heteroarylhalogenide, diisopropylethylamine (3 eq) were added in this sequence and the reaction mixture was heated at 80 to 90° C. for 3 to 5 hours.

The solvent was removed in vacuo and the residue was dissolved in ethylacetate. The organic layer was extracted 3 times with 1 M citric acid, aqueous potassium hydrogen carbonate and 2 times with brine, dried over MgSO₄ and filtred. The solvent was removed and the residue was purified by silica gel chromatography using a DCM/MeOH 95:5 gradient. Yield: 40-80%

Exemplary compounds of formula (Ihb) of the present invention include, but are not limited to, the following:

| | | | LC/(+)-ESI- | Biological |
|---|---|---|---|---|
| Cp. | Name | Mass | MS: [M + H]⁺ | activity |
| 1 | N-(6-benzoyl-1H-benzo[d]imidazol-2-yl)- | 569 | 570 | +++ |
| | 2-(1-(2,6-dimethoxypyrimidin-4- | | | |
| | yl)piperidin-4-yl)thiazole-4-carboxamide | | | |
| 2 | 2-(1-(7H-purin-6-yl)piperidin-4-yl)-N-(6- | 549 | 550 | +++ |
| | benzoyl-1H-benzo[d]imidazol-2- | | | |
| | yl)thiazole-4-carboxamide | | | |
| 3 | N-(6-benzoyl-1H-benzo[d]imidazol-2-yl)- | 533 | 534 | + |
| | 2-(1-(4-cyanopyridin-2-yl)piperidin-4- | | | |
| | yl)thiazole-4-carboxamide | | | |
| 4 | N-(6-benzoyl-1H-benzo[d]imidazol-2-yl)- | 565 | 566 | +++ |
| | 2-(1-(thieno[3,2-d]pyrimidin-4- | | | |
| | yl)piperidin-4-yl)thiazole-4-carboxamide | | | |
| 5 | N-(6-benzoyl-1H-benzo[d]imidazol-2-yl)- | 577 | 578 | ++ |
| | 2-(1-(6-(trifluoromethyl)pyrimidin-4- | | | |
| | yl)piperidin-4-yl)thiazole-4-carboxamide | | | |
| 6 | N-(5-benzoyl-1H-benzo[d]imidazol-2-yl)- | 746 | 747 | +++ |
| | 2-(1-(6-methoxy-7-(3-(4-methylpiperazin- | | | |
| | 1-yl)propoxy)quinazolin-4-yl)piperidin-4- | | | |
| | yl)thiazole-4-carboxamide | | | |
| 7 | N-(5-benzoyl-1H-benzo[d]imidazol-2-yl)- | 459 | 460 | +++ |
| | 2-(1-formylpiperidin-4-yl)thiazole-4- | | | |
| | carboxamide | | | |
| 8 | Methyl 4-(4-(4-(5-benzoyl-1H- | 420 | 421 | + |
| | benzo[d]imidazol-2-ylcarbamoyl)thiazol-2- | | | |
| | yl)piperidin-1-yl)-5-methylpyrrolo[1,2- | | | |
| | f][1,2,4]triazine-6-carboxylate | | | |
| 9 | N-(5-benzoyl-1H-benzo[d]imidazol-2-yl)- | 562 | 563 | +++ |
| | 2-(1-(2-methylpyrazolo[1,5-a]pyrimidin-7- | | | |
| | yl)piperidin-4-yl)thiazole-4-carboxamide | | | |
| 10 | 2-(1-([1,2,4]triazolo[1,5-a]pyrimidin-7- | 549 | 550 | +++ |
| | yl)piperidin-4-yl)-N-(5-benzoyl-1H- | | | |
| | benzo[d]imidazol-2-yl)thiazole-4- | | | |
| | carboxamide | | | |
| 11 | Methyl 7-(4-(4-(5-benzoyl-1H- | 607 | 608 | +++ |
| | benzo[d]imidazol-2-ylcarbamoyl)thiazol-2- | | | |
| | yl)piperidin-1-yl)-[1,2,4]triazolo[1,5- | | | |
| | a]pyrimidine-2-carboxylate | | | |
| 12 | N-(6-benzoyl-1H-benzo[d]imidazol-2-yl)- | 619 | 620 | +++ |
| | 2-(1-(2,6-dichloropyridin-4- | | | |
| | ylcarbamoyl)piperidin-4-yl)thiazole-4- | | | |
| | carboxamide | | | |
| 13 | N-(5-benzoyl-1H-benzo[d]imidazol-2-yl)- | 570 | 571 | +++ |
| | 2-(1-(4,6-dimethoxy-1,3,5-triazin-2- | | | |
| | yl)piperidin-4-yl)thiazole-4-carboxamide | | | |
| 14 | N-(5-benzoyl-1H-benzo[d]imidazol-2-yl)- | 619 | 620 | +++ |
| | 2-(1-(6,7-dimethoxyquinazolin-4- | | | |
| | yl)piperidin-4-yl)thiazole-4-carboxamide | | | |
| 15 | N-(5-benzoyl-1H-benzo[d]imidazol-2-yl)- | 553 | 554 | +++ |
| | 2-(1-(3-nitropyridin-4-yl)piperidin-4- | | | |
| | yl)thiazole-4-carboxamide | | | |
| 16 | N-(5-benzoyl-1H-benzo[d]imidazol-2-yl)- | 619 | 620 | +++ |
| | 2-(1-(2- | | | |
| | (trifluoromethoxy)benzoyl)piperidin-4- | | | |
| | yl)thiazole-4-carboxamide | | | |
| 17 | N-(5-benzoyl-1H-benzo[d]imidazol-2-yl)- | 605 | 606 | +++ |
| | 2-(1-(4,7-dimethylpyrazolo[5,1- | | | |
| | c][1,2,4]triazine-3-carbonyl)piperidin-4- | | | |
| | yl)thiazole-4-carboxamide | | | |

¹⁾The biological data refer to results obtained from the NF-κB inflammation assay.
[“+” stands for an EC50 of 30-100 μM, “++” stands for an EC50 of 10-30 μM and “+++” stands for an EC50 of smaller than 10 μM.]

### Inhibition of NF-κB-Induced Inflammation:

For the determination of anti-inflammatory activity of the compounds the PRINCESS® NINA Instant Assay from Cell Culture Service GmBH was used. This assay is based on recombinant A549—NF-κB-SEAP reporter cells preseeded in 96-well flat bottom plates. As the transfected reporter gene for SEAP (secreted embryonic alkaline phosphatase) is under transcriptional control of a NF-κB-responsive element, the expression of this reporter is activated upon stimulation with TNF-α. SEAP secretion into the culture supernatant can be detected by the chemiluminescent substrate CSPD®. Test compounds that inhibit the NF-κB activation show reduced SEAP activity and reduced luminescent readout.

Following 18 h of reactivation at 37° C., 5% CO₂ and 90% relative humidity, the cells were incubated with 0.01 up to 100 μM of test compound for 4.5 h before stimulation with 2 ng/ml TNF-α. After stimulation with TNF-α for 22 h endogenous phosphatases were inactivated and CSPD® substrate was supplied for 40 min. SEAP activity then was quantified by measuring luminescence as relative light units (RLU) using a Tecan Ultra reader. Each data point was recorded in quadruplicates and EC50 values were calculated via fitting function and the Microsoft Excel Solver.

### T-Lymphocyte Proliferation Assay:

Inhibition of stimulated peripheral blood monocytes (PBMC).

PBMCs were isolated from blood of healthy volunteers with the help of ACCUSPIN™ System Histopaque®-1077 tubes, washed and resuspended with 10⁶ cells/ml in RPMI1640-Glutamax-Medium, containing 10% fetal calf serum, 4 mM L-Glutamine, 100 units/ml Penicillin and 100 μg/ml Streptomycin. The cells were stimulated with phytohemoagglutinin in the presence of test compound or blank vehicle for 48 hours. 4 h prior to the end of the incubation period, 5-bromo-2′-desoxyuridine (BrdU) was added to label the proliferating cells. After the incubation, the cells were separated by centrifugation and the culture supernatant removed. Incorporated BrdU was quantified with the help of an enzyme-linked immunosorbent assay. For the determination of the IC₅₀ values (concentration of inhibitor required for 50% inhibition) at least four different inhibitor concentrations were applied. Each data point was recorded in triplicates. Curves were fitted with the a suitable program.

Based on results obtained in the T-lymphocyte proliferation assay, the compounds of the present invention are suitable for treating inflammatory diseases or diseases associated with T-cells.

### Analysis of Cytokine Production of Human PBMCs:

PBMCs were isolated as for T-lymphocyte proliferation assay, washed and resuspended with 10⁶ cells/ml in RPMI1640-Glutamax-Medium, containing 10% fetal calf serum and 4 mM L-Glutamine, 100 units/ml Penicillin and 100 μg/ml Streptomycin. The cells were treated with 2.5 or 25 μM compound or the same amount of DMSO as negative control and were then stimulated with one of the following stimuli: 2 μg/ml phytohemeagglutinin, 10 ng/ml IL-1beta, 10 ng/ml TNF-alpha, 2.5 μg/ml CL075, 1 μg/ml Lypopolysaccharide (LPS), 1 μM ODN2006 or 1 μM ODN1668. The cytokine production was analysed in supernatant after 24, 48 and 72 hours using luminex bioplex system (BioRad, Munich, Germany) according to the manufacturer's instructions.

Oligonucleotides with the sequences 5′-TsCsCsAsTsGsAsCsGsTsTsCsCsTsGsAsTsGsCsTs-3′ (ODN1668) and 5′-GsGsGGGACGATCgTCGsGsGsGsGsG-3′ were synthesised by TiB-Molbiol (Berlin, Germany), s depicts a phosphorothiate linkage. LPS and PHA were obtained from Sigma-Aldrich (Taufkirchen, Germany), IL-1-beta and TNF-alpha from Strathman Biotec GmbH (Hamburg, Germany).

### Analysis of Cytokine Production of Mouse Splenocytes:

Splenocytes were isolated from female BALB/CanNCrl mice (Charles River Laboratories), washed and resuspended with 2×10⁶ cells/ml in splenocyte medium (RPMI-medium, 5% heat-inactivated fetal calf serum, 20 mM HEPES, 50 μM 2-mercapto-ethanol, 1% PEN/Step solution (PAN Biotech)). Cells were treated with test substances or DMSO as control and seeded into CD3-coated or uncoated 96-well plates. Cells in CD3-coated plates were further stimulated with anti-CD28. After incubation for 24 or 48 hours at 37° C., wells were harvested and cell-free culture supernatants were frozen at −80° C. until measurement of marker cytokines by Luminex technology.

Analysis of Influenza Virus Specific Nucleic Acid and IL-6 and IP-10 mRNA in H5N1 Bird Flu Virus Infected Mouse Lung Tissue:

Mice were infected with bird flu virus H5N1 (strain MB1) and treated for two days with compound 4 (daily 15 mg/kg i.p.). After two days mice were sacrificed and RNA extracted from the lung tissue. Influenza virus specific real time PCR was performed with the artus Qiagen RT-PCR kit. Cytokine/chemokine specific real time PCR was performed with the Qiagen RT-PCR kit.

### Inhibition of Influenza Virus Replication

A549 cells were infected with highly pathogenic avian influenza virus strains (e.g. H5N1, H7N7, H2N3) or various strains of human influenza virus (e.g. H1N1) for 16 hours (MOI=0.001). Cells were incubated with different concentrations of compounds or solvent (DMSO). The titer of next generation virus was determined in plaque assays. The virus number of the DMSO control was defined as 100%.

### Determination of Cell Viability:

Cell lines (e.g. A549) incubated with compounds of the invention were assessed on cell viability by means of MTT staining.

### Analysis of Resistance Phenomena:

A549 cells were infected with fowl plaque virus (influenza strain H7N7), MOI=0.01 and incubated for 24 hours in the presence or absence of substance. The supernatant of each sample was collected and viral titers determined by plaque assays on MDCK cells. Subsequently, the supernatants were normalized and a second cell passage of A549 cells was infected with equal numbers of virus under the same conditions as in the first passage. This procedure was repeated to the 5^{th} cell passage.

### Influenza Mouse Models:

Female Balb/C or C57BI/6 mice were infected intra-nasally with LD50 doses of influenza viruses. Compounds were applied once or twice daily i.v. or i.p. starting on the day of infection (day 0) or 4 days after infection for several days. The number of surviving mice from treatment groups was compared at day 14 to infected, but untreated control groups. For confirmation of infection virus antibody titers were determined for all animals.

### Cancer Cell Experiments:

The 23 cell lines were purchased from ATCC (Rockville, Md.), DSMZ (Braunschweig, Germany) or ECACC (Wiltshire, UK). Cells were routinely passaged once or twice weekly. They were maintained in culture for no more than 20 passages. All cells were grown at 37° C. in a humidified atmosphere (95% air, 5% CO₂) in RPMI 1640 medium (PAA, Cölbe, Germany) supplemented with 10% fetal calf serum (PAA, Colbe, Germany) and 0.1% gentamicin (PAA, Cölbe, Germany).

A modified propidium iodide assay was used to assess the substance's anticancer activity. Briefly, cell suspension was taken from exponential phase cultures, counted and plated in 96 well flat-bottomed microtiter plates at a cell density depending on the cell line (20.000-100.000 cells/well). After a 24 h recovery period allowing cells to resume exponential growth, 10 μl of culture medium (6 control wells/plate) or culture medium containing the test compound was added to the cells. Test articles were applied in triplicates at 5 concentrations, ranging from 0.003 to 30 μM. Following 4 days of continuous drug exposure, 50 μl of an aqueous propidium iodide (PI) solution was added to the wells (final PI concentration of 7 μg/ml). PI does not cross intact cell membranes and enters only the nucleus of dead cells. After a 30 min incubation period fluorescence (FU1) was measured using a Cytofluor 4000 microplate reader (excitation 530 nm, emission 620 nm) giving a direct measurement of dead cells. Microplates were then kept at −20° C. for 24 hrs, resulting in a total cell kill. After thawing of the plates and a second fluorescence measurement (FU2) the amount of viable cells was calculated by substraction of FU1 from FU2.

All compound/test cell line combinations were tested in 3-4 independent experiments. In each experiment, all data points were determined in triplicate. For calculations the mean of triplicate data was used.

Growth inhibition was expressed as the ratio of fluorescence of test and control wells (Test/Control×100 [% T/C]). Based on the T/C values IC50/IC70/IC90 values, being the drug concentrations necessary to inhibit cell growth by 50% (T/C=50%), 70% (T/C=30%) and 90% (T/C=10%), respectively, were calculated by plotting compound concentration versus cell viability (T/C) according the principle of point-to-point curve fit. Mean IC50- and IC70-values are described by the equation

$$mean {IC}_{50 , 70} = {10}^{( \frac{{\sum }_{x = 1}^{n} log ( {IC}_{50 , 70} )}{n} )}$$

with x representing the specific tumor cell line and n the total number of tumor cell lines studied. If an IC50- or IC70-value could not be determined within the examined concentration range (because a compound was either too active or lacked activity), the lowest or highest concentration studied was used for the calculation.

Antitumor activity and tumor selectivity were evaluated using the IC50 or IC70 mean graph presentations. They show the distribution of the individual IC50/IC70 values for each cell line related to the mean IC50/IC70-values. Deviations of individual IC50/IC70-values from the mean IC50/IC70-value are represented as bars on a logarithmically scaled axis. Bars to the left represent IC50/IC70-values lower than the mean IC70 (sensitive cell lines), bars to the right show higher individual IC50/IC70-values (resistant cell lines).

### BRIEF DESCRIPTION OF THE DRAWINGS

FIG. 1: Compounds, e.g. compound 4 (at 10 μM concentration), inhibits relevant cytokine production in mouse splenocytes stimulated with CD3/CD28; all measured cytokines were completely inhibited after 24 hours, and after 48 hours secretion of most cytokines was still reduced by more than 90% of the DMSO control.

FIGS. 2 and 3: In human PBMCs stimulated with IL-1beta, secretion of cytokines like IL-6 and IL-8 are blocked by compound 4 (cpd) dose dependently.

FIGS. 4 and 5: In human PBMCs stimulated with ODN2216, secretion of cytokines like IL-8 and IFN-gamma are blocked by compound 4 (cpd) dose dependently.

FIGS. 6 and 7: In human PBMCs stimulated with ODN1668, secretion of cytokines like IL-6 and IL-8 are blocked by compound 4 (cpd) dose dependently.

FIGS. 8-12: In human PBMCs stimulated with CL075, secretion of cytokines like IL-1beta, IL-6, IL-8, IFN-gamma, and TNF-alpha are blocked by compound 4 (cpd) dose dependently.

FIGS. 13-15: In human PBMCs stimulated with TNF-alpha, secretion of cytokines like IL-1beta, IL-6, and IL-8 are blocked by compound 4 (cpd) dose dependently.

FIGS. 16-21: In human PBMCs stimulated with LPS, secretion of cytokines like IL-8, IL-1beta, INF-gamma, TNF-alpha, IL-10, and IL-6 are blocked by compound 4 (cpd) dose dependently.

FIGS. 22-29: In human PBMCs stimulated with PHA, secretion of cytokines like IL-1beta, IL-6, IL-2, IL-8, IL-10, IL-12p70, INF-gamma, and TNF-alpha are blocked by compound 4 (cpd) dose dependently.

FIGS. 30-32: The influence of various concentrations of compound 4 on cytokine release was measured 48 hours after stimulation. After stimulation with PHA, IL-6, TNF-alpha and INF-gamma secretion was inhibited by compound 4 with EC50 values of 0.2 μM, 7.9 μM and 9.0 μM, respectively.

FIGS. 33 and 34: The influence of various concentrations of compound 4 on cytokine release was measured 48 hours after stimulation. After stimulation with LPS, IL-6 and TNF-alpha secretion was inhibited by compound 4 with EC50 values of 0.6 μM and 12.6 μM, respectively.

FIGS. 35-37: Inhibition of production of H5N1 specific nucleic acid and mRNA for IL-6 and IP-10 was analysed in lung tissue from influenza virus (H5N1) infected mice in the presence and absence of compound 4. Compound 4 leads to reduced expression of H5N1 specific mRNA in the lung of infected mice and also to reduced transcription of IL-6 and IP-10 genes.

FIG. 38: Analysis of anti-viral effects of compounds on virus proliferation.

Virus titers of Influenza A virus in infected A549 cells are reduced by 5 μM compound 4 (KH1) by 4 log units (H5N1-strain) and by 5 log units (Fowl Plaque Virus, H7N7-strain), respectively.

H5N1: shown is a logarithmic representation of the virus titers in relation to the untreated control of different concentrations of compound 4 (KH1).

FIG. 39: H7N7: shown is a logarithmic representation of the virus titer in relation to the untreated control of different concentrations of compound 4 (KH1).

FIG. 40: Determination of cell viability after treatment with compound 4:

A549 cells were treated with 5 μM compound 4 (KH1) in the absence of virus. Cell survival was examined by MTT staining. It is shown that cell viability (% Zellüberleben) over the tested time range of 96 hours is the same for cells treated with compound 4 (KH1) as for DMSO treated cells (DMSO) and untreated cells (unbeh) thus demonstrating that the compounds of the invention do not exert toxic effects on the cells.

FIGS. 41-42: Multi-passanging experiment for the detection of resistance phenomena:

Compound 4 (KH1; 5 μM), amantadine (5 μM), and oseltamivir (2 μM) versus an untreated control (FPV) were examined in a multi-passaging experiment for the detection of resistance phenomena. In contrast to amantadine and oseltamivir, compound 4 does not show the induction of resistance mechanisms and development of viral resistance after 5 cell passanging cycles (two independent experiments are shown).

FIG. 43: Influenza mouse models with H7N7:

Mice were infected with LD50 of influenza strain H7N7 on day 0. Treatment was started on the same day with 15 mg/kg compound 4 daily i.p. Treatment was stopped on day 8 and the experiment terminated on day 14. Results of this experiment displayed complete protection of infected mice as compared to the control group in which half of the animals died (p<0.05).

FIGS. 44-46: Mice were infected with LD50 of influenza strain H7N7 on day 0. Treatment was started on the same day with various doses of compound 4 daily i.v. Treatment was stopped on day 4 and the experiment terminated on day 14. Results of this experiment displayed significant protection of mice (p<0.05) when compound 4 is given at doses equal or higher 5 mg/kg.

FIG. 47: Influenza mouse models with H5N1:

Mice were infected with LD50 of influenza strain H5N1 on day 0. Treatment was started on the same day with two times 7.5 mg/kg compound 4 daily i.p. Treatment was stopped on day 10 and the experiment terminated on day 14. Results of this experiment displayed significant protection of mice (p<0.05).

FIGS. 48-49: Mice were infected with LD50 of influenza strain H5N1 on day 0. Treatment was started on day 4 with 7.5 mg/kg compound 4 b.id. and 15 mg/kg compound 4 daily i.p. Treatment was stopped on day 10 and the experiment terminated on day 14. Results of this experiment displayed significant protection of mice (p=0.05).

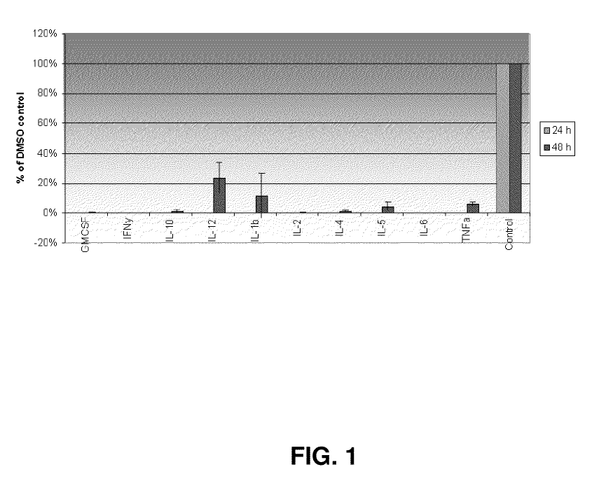

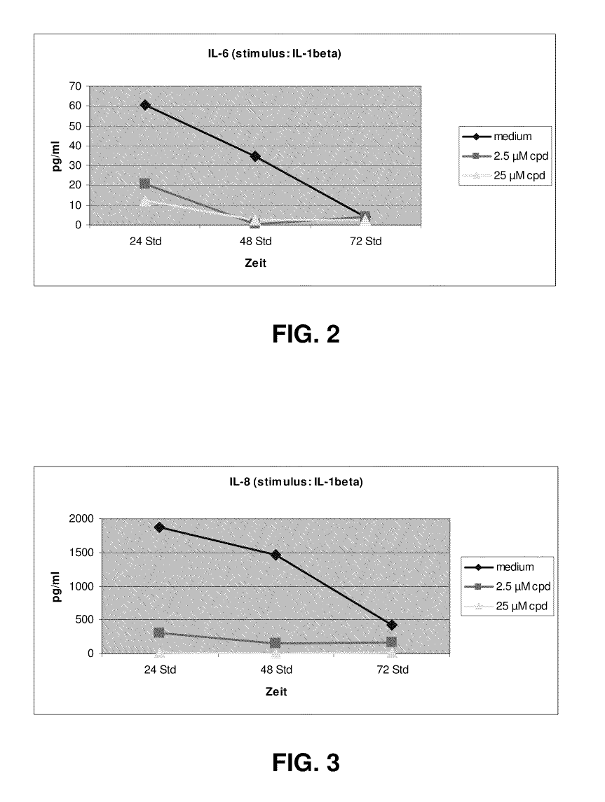

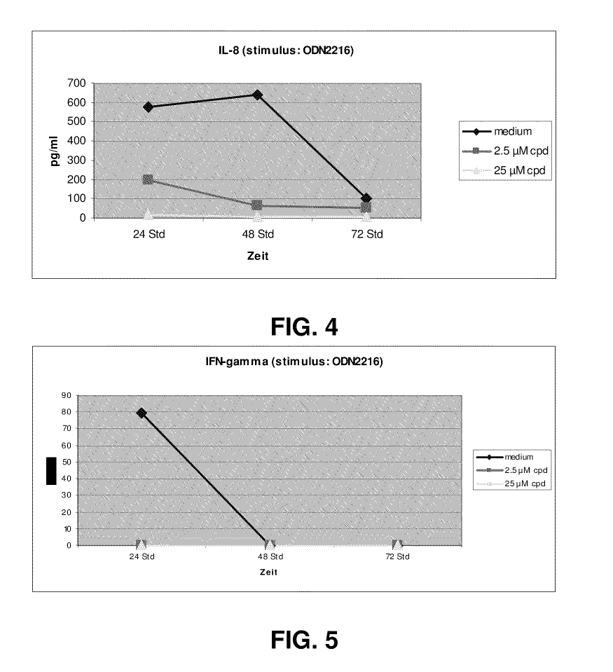

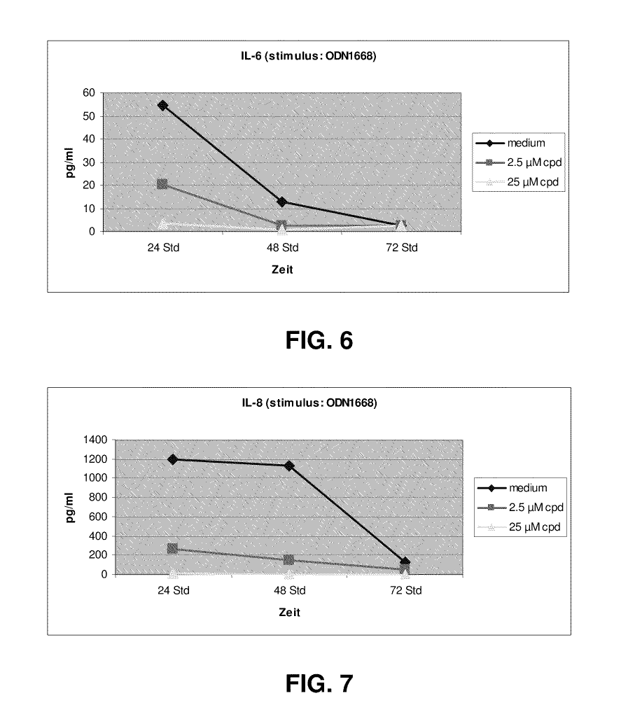

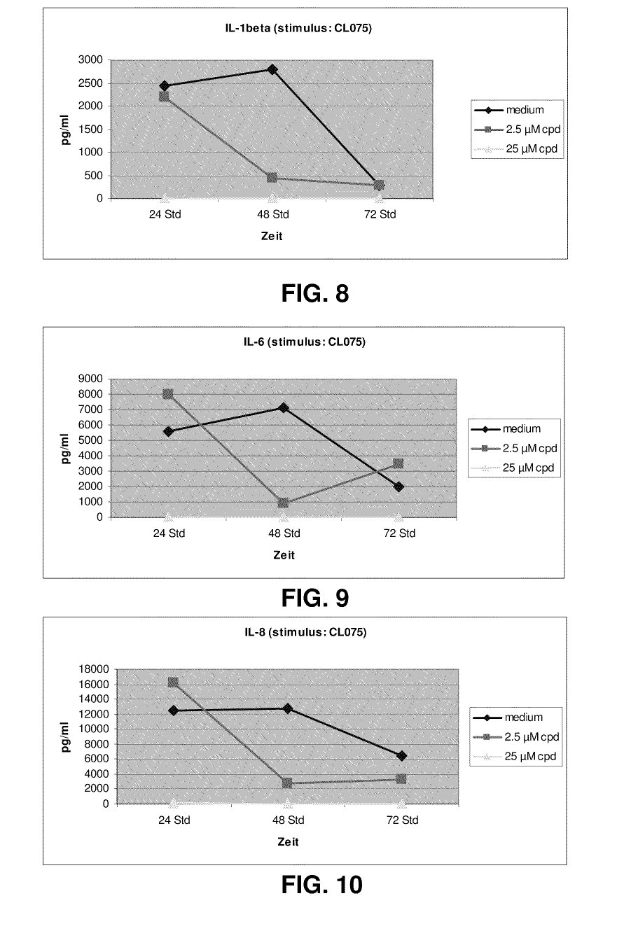

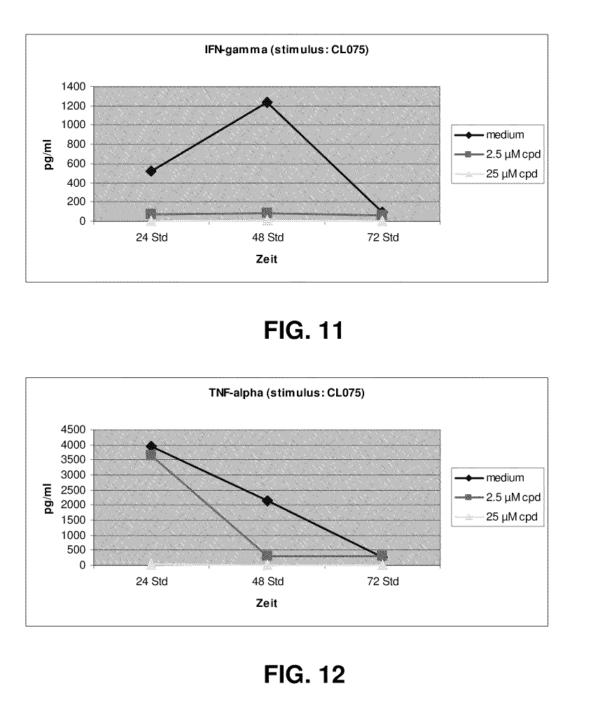

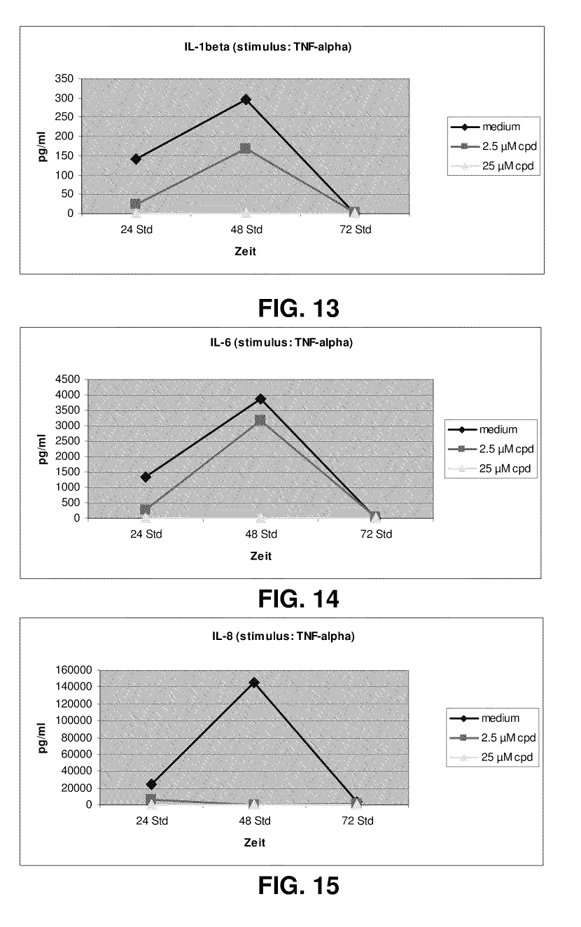

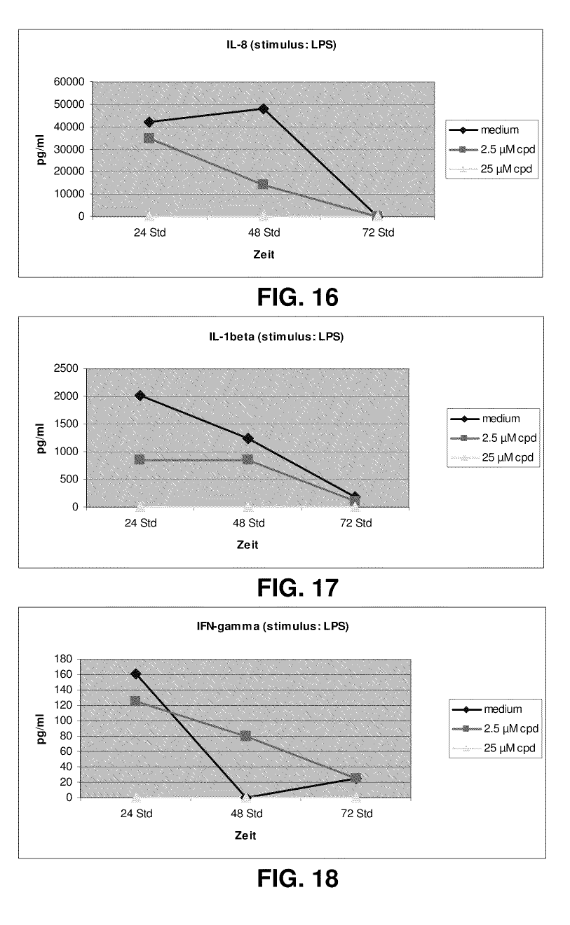

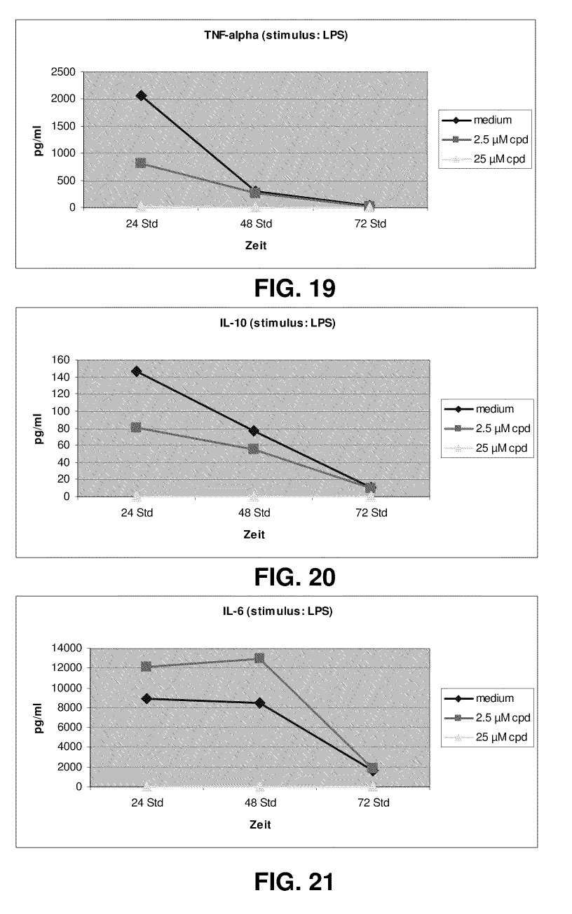

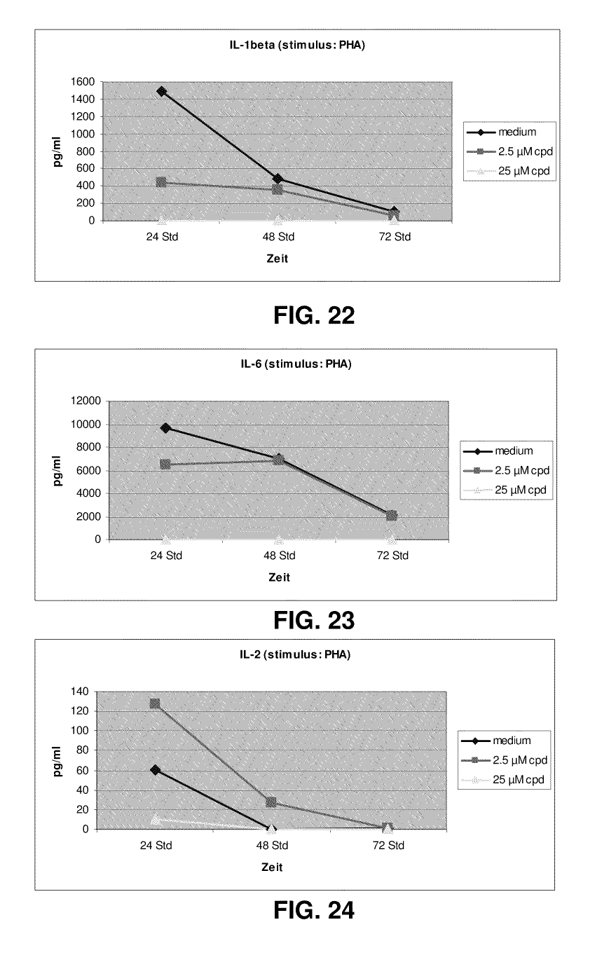

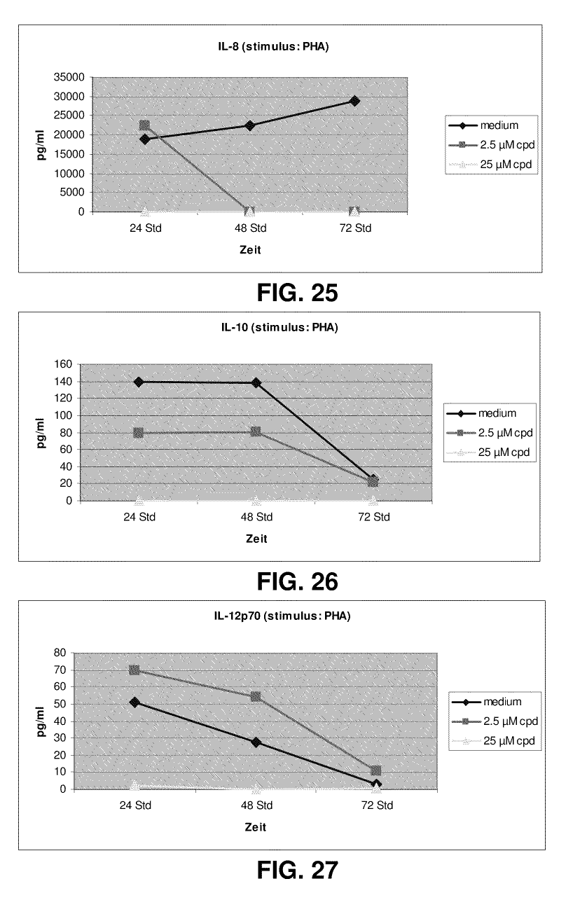

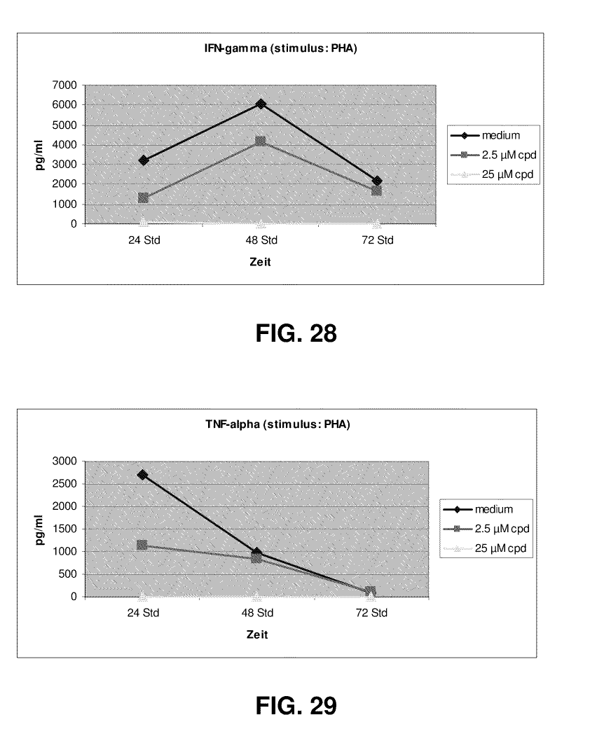

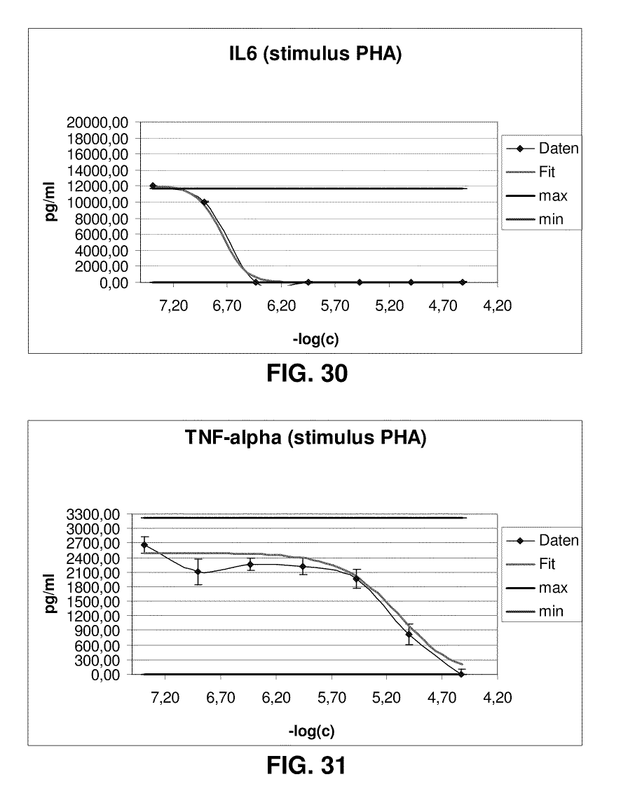

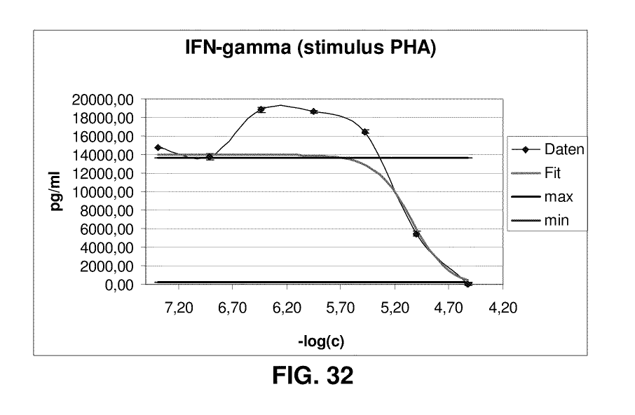

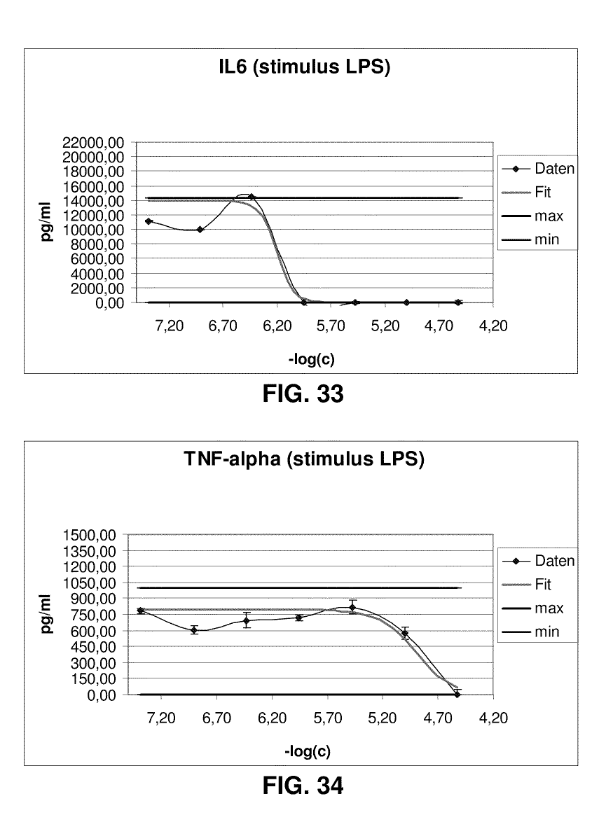

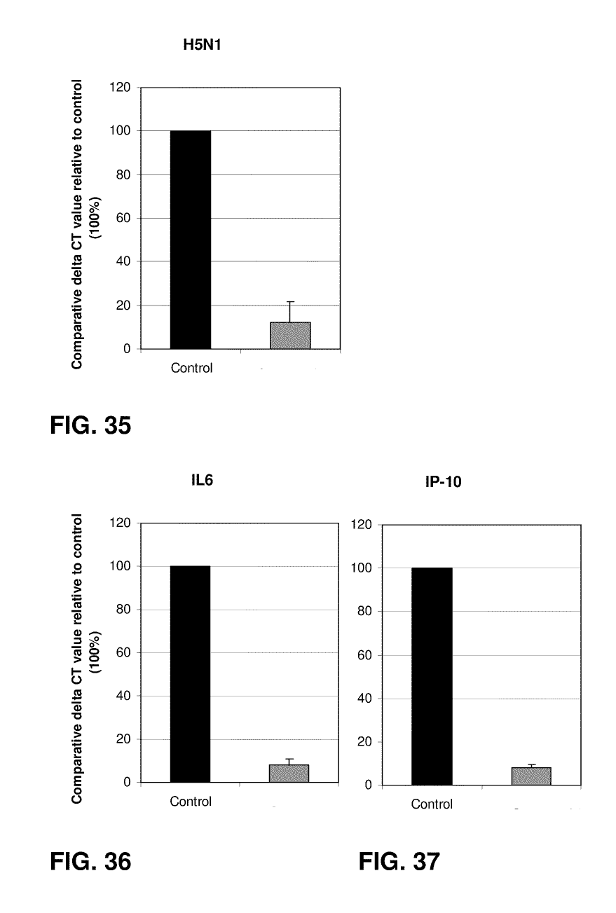

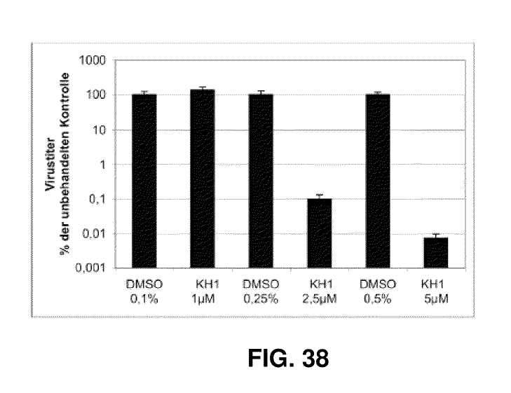

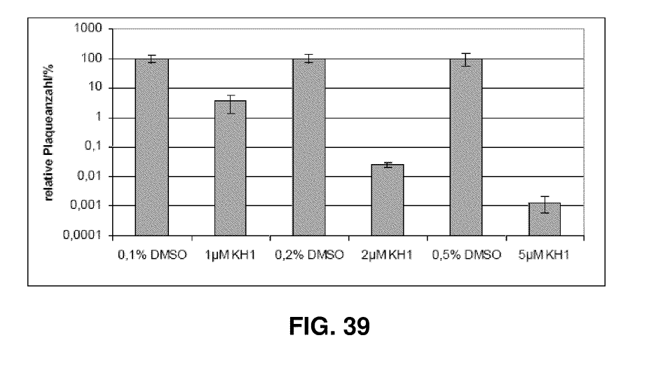

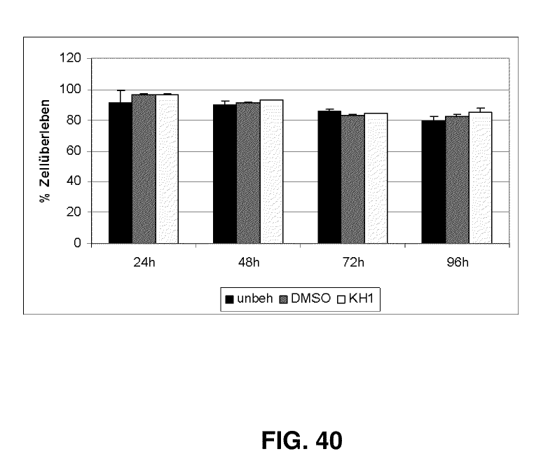

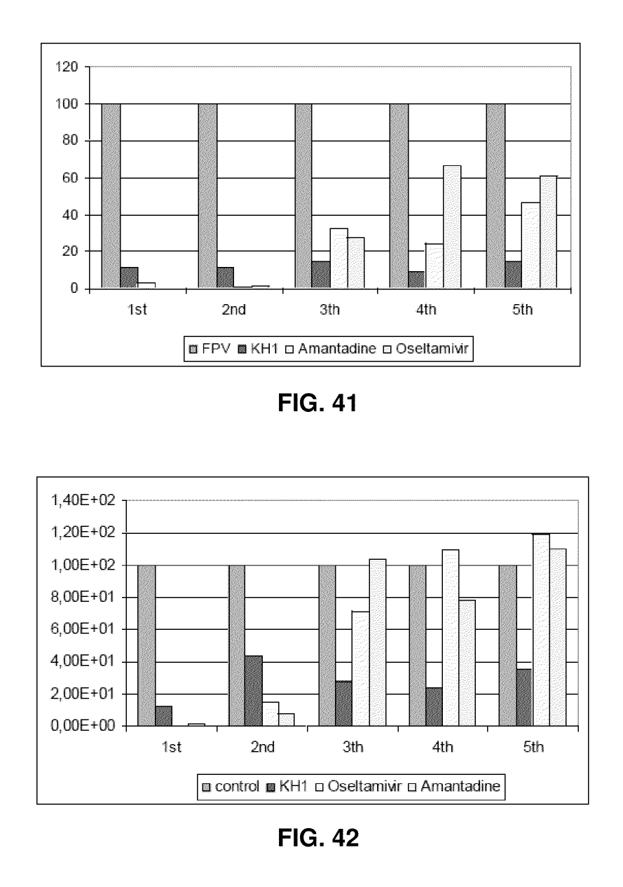

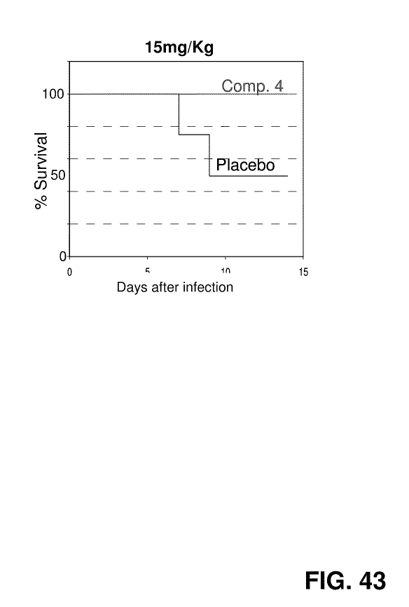

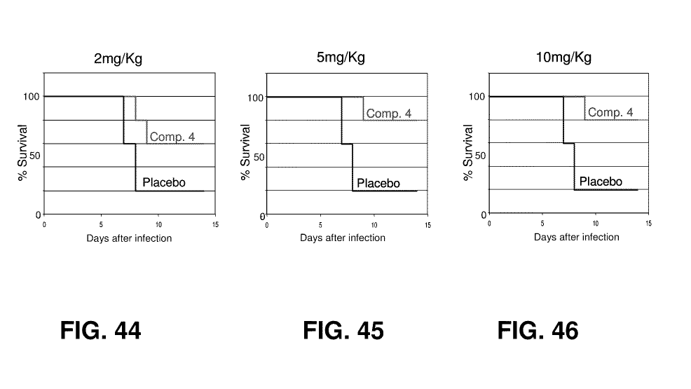

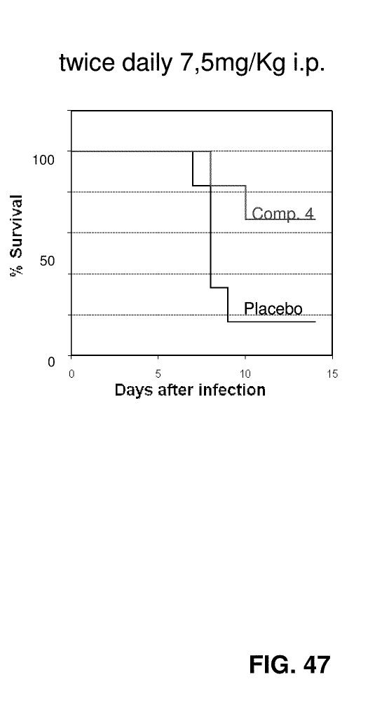

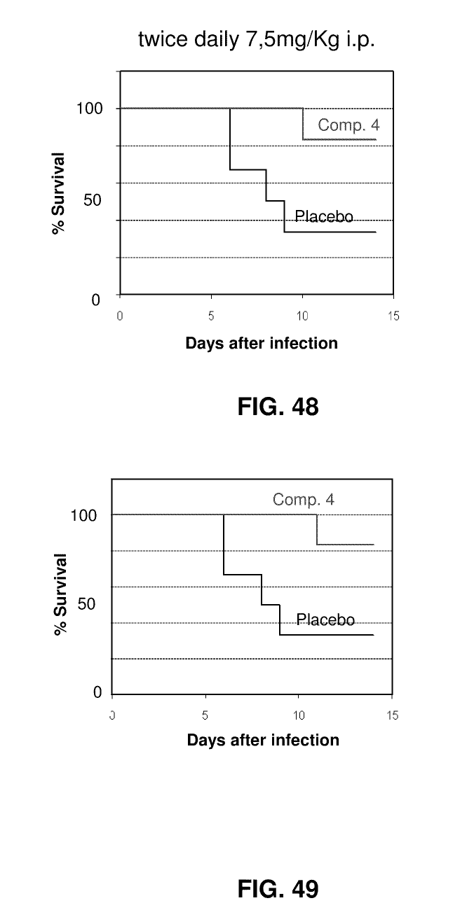

### Cancer Cell Data:

Compound 4 was tested on a panel of hematological cell lines for its anti-cancer activity. It displayed an excellent score of tumor sensitivity. Predominantly cell lines derived from multiple myelomas (4 out of 5) were above average sensitive.

IN-VITRO ANTITUMOR ACTIVITY OF compound 4 IN HUMAN TUMOR CELL LINES (Monolayer Assay, PI)

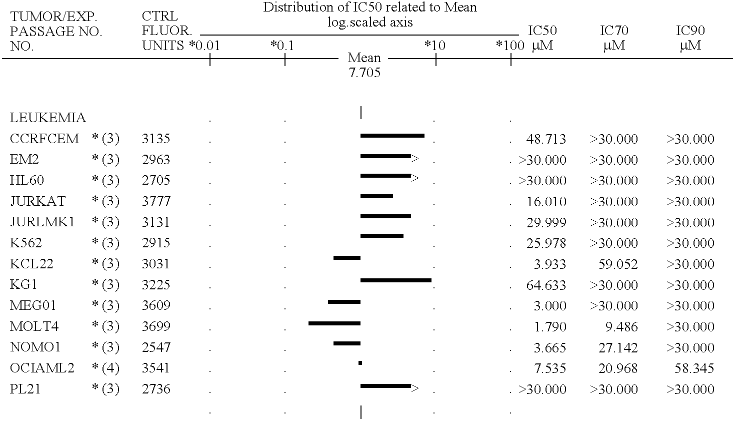

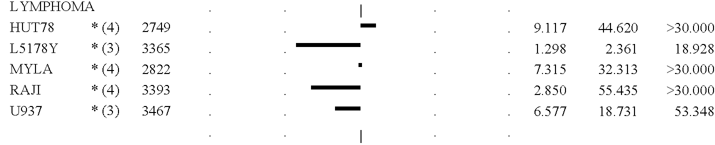

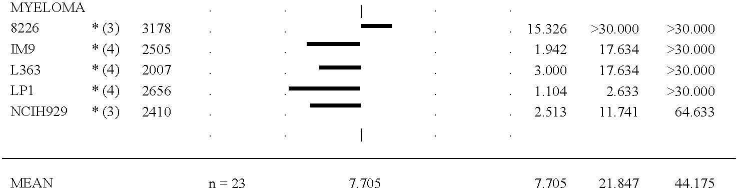

Without further elaboration, it is believed that one skilled in the art can, using the preceding description, utilize the present invention to its fullest extent. The preceding preferred specific embodiments are, therefore, to be construed as merely illustrative, and not limitative of the remainder of the disclosure in any way whatsoever.

In the foregoing and in the examples, all temperatures are set forth uncorrected in degrees Celsius and, all parts and percentages are by weight, unless otherwise indicated.

The entire disclosures of all applications, patents and publications, cited herein and of corresponding European application No. 07116791.0, filed Sep. 19, 2007, are incorporated by reference herein.

The preceding examples can be repeated with similar success by substituting the generically or specifically described reactants and/or operating conditions of this invention for those used in the preceding examples.

From the foregoing description, one skilled in the art can easily ascertain the essential characteristics of this invention and, without departing from the spirit and scope thereof, can make various changes and modifications of the invention to adapt it to various usages and conditions.

1. A compound of the general formula (Ihb) or pharmaceutically acceptable salts thereof with an acid or a base, or a stereoisomer thereof for the prevention and/or treatment of a disease associated with increased cytokine release, 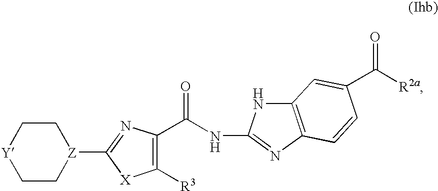
wherein
Y′ is O or NR^{2′};
z is N or CR^{2′};
X is NR^{2′}, O or S;
R^{2′} is H, alkyl, —C(O)NR⁷, —C(O)R^{e}, cycloalkyl, haloalkyl, hydroxyalkyl, hydroxyalkylamino, alkylamino, heteroaryl, or aryl;
R³ is H, methyl, ethyl, methoxy, amine, alkylamine, morpholino, N-methylpiperazine, CF₃, or OCF₃;
R^{2a} is substituted or unsubstituted aryl, benzyl or heteroaryl;
R⁷, R⁷, R⁸ independently are H, halogen, alkyl, cycloalkyl, heterocycloalkyl, haloalkyl, hydroxyalkyl, hydroxyalkylamino, alkylamino, arylamino, heteroaryl, or aryl;
R^{e} independently reperesents H, —CN, —OH, —SH, —CO₂R^{4′}, —C(O)R^{4′}, —SO₂R^{4′}, —NR^{4′}R^{5′}, —C(O)NR⁷R⁸, —SO₂-alkyl, —SO₂R^{4′}, —SO₃R^{4′}, —N═CR^{4′}R^{5′}, —NR^{4′}C(O)R^{4″}, —NR^{4′}-CO-haloalkyl, —NO₂, —NR^{4′}—SO₂-haloalkyl, —NR^{4′}—SO₂-alkyl, —NR^{4′}—CO-alkyl, —NR^{4′}(CH₂)ₚheteroaryl, alkyl, hydroxyalkyl, cycloalkyl, alkylamino, aryl, hydroxyalkylamino, alkoxy, alkylthio, —O(CH₂)ₚ[O(CH₂)ₚ]_{q}OCH₃, —C(NR^{4″})NR^{4′}-benzimidazolyl, —C(NR^{4″})NR^{4′}-benzthiazolyl, —C(NR^{4″})NR^{4′}-benzoxazolyl, or heteroaryl;
R^{4′}, R^{4″}, R^{5′} independently are H, halogen, alkyl, —C(NR⁷)NR⁷R⁸, —(CH₂)ₚaryl, haloalkyl, —(CH₂)ₚNR⁷R⁸, —C(O)NR⁷R⁸, —N═CR⁷R⁸, —NR⁷C(O)R⁸, cycloalkyl, heterocycloalkyl, hydroxyalkyl, hydroxyalkylamino, alkylamino, heteroaryl, or aryl.
p=1-6
q=1-6

2. A compound of the general formula (Ihb-2) or pharmaceutically acceptable salts thereof with an acid or a base, or a stereoisomer thereof for the prevention and/or treatment of a disease associated with increased cytokine release, 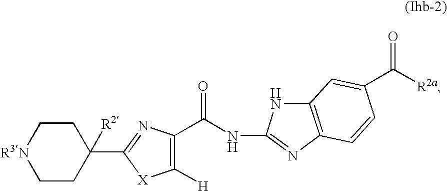
wherein
R^{3′} is substituted or unsubstituted heteroaryl or aryl.
X is NR^{2′}, O or S;
R^{2′} is H alkyl, —C(O)NR⁷, —C(O)R^{e}, cycloalkyl, haloalkyl, hydroxyalkyl, hydroxyalkylamino, alkylamino, heteroaryl, or aryl;
R^{2a} is substituted or unsubstituted aryl or heteroaryl;
R⁷, R^{7′}, R⁸ independently are H, halogen, alkyl, cycloalkyl, heterocycloalkyl, haloalkyl, hydroxyalkyl, hydroxyalkylamino, alkylamino, arylamino, heteroaryl, or aryl;
R^{e} independently reperesents H, —CN, —OH, —SH, —CO₂R^{4′}, —C(O)R^{4′}, —SO₂R^{4′}, —NR^{4′}R^{5′}, —C(O)NR⁷R⁸, —SO₂-alkyl, —SO₂R^{4′}, —SO₃R^{4′}, —N═CR^{4′}R^{5′}, —NR^{4′}C(O)R^{4″}, —NR^{4′}-CO-haloalkyl, —NO₂, —NR^{4′}—SO₂-haloalkyl, —NR^{4′}—SO₂-alkyl, —NR^{4′}—CO-alkyl, —NR^{4′}(CH₂)ₚheteroaryl, alkyl, hydroxyalkyl, cycloalkyl, alkylamino, aryl, hydroxyalkylamino, alkoxy, alkylthio, —O(CH₂)ₚ[O(CH₂)ₚ]_{q}OCH₃, —C(NR^{4″})NR^{4′}-benzimidazolyl, —C(NR^{4″})NR^{4′}-benzthiazolyl, —C(NR^{4″})NR^{4′}-benzoxazolyl, or heteroaryl;
R^{4′}, R^{4″}, R^{5′} independently are H, halogen, alkyl, —C(NR⁷)NR^{7′} R⁸, —(CH₂)ₚaryl, haloalkyl, —(CH₂)ₚNR⁷R⁸, —C(O)NR⁷R⁸, —N═CR⁷R⁸, —NR⁷C(O)R⁸, cycloalkyl, heterocycloalkyl, hydroxyalkyl, hydroxyalkylamino, alkylamino, heteroaryl, or aryl.
p=1-6
q=1-6

3. A compound of the general formula (Ihb-3) or pharmaceutically acceptable salts thereof with an acid or a base, or a stereoisomer thereof for the prevention and/or treatment of a disease associated with increased cytokine release, 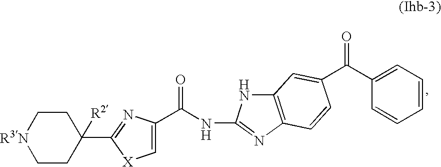
wherein
R^{3′} is a substituted or unsubstituted bicyclic heteroaryl;
X is NR^{2′}, O or S;
R^{2′} is H, alkyl, —C(O)NR⁷, —C(O)R^{e}, cycloalkyl, haloalkyl, hydroxyalkyl, hydroxyalkylamino, alkylamino, heteroaryl, or aryl;
R⁷, R^{7′}, R⁸ independently are H, halogen, alkyl, cycloalkyl, heterocycloalkyl, haloalkyl, hydroxyalkyl, hydroxyalkylamino, alkylamino, arylamino, heteroaryl, or aryl;
R^{e} independently reperesents H, —CN, —OH, —SH, —CO₂R^{4′}, —C(O)R^{4′}, —SO₂R^{4′}, —NR^{4′}R^{5′}, —C(O)NR⁷R⁸, —SO₂-alkyl, —SO₂R^{4′}, —SO₃R^{4′}, —N═CR^{4′}R^{5′}, —NR^{4′}C(O)R^{4″}, —NR^{4′}-CO-haloalkyl, —NO₂, —NR^{4′}—SO₂-haloalkyl, —NR^{4′}—SO₂-alkyl, —NR^{4′}—CO-alkyl, —NR^{4′}(CH₂)ₚheteroaryl, alkyl, hydroxyalkyl, cycloalkyl, alkylamino, aryl, hydroxyalkylamino, alkoxy, alkylthio, —O(CH₂)ₚ[O(CH₂)ₚ]_{q}OCH₃, —C(NR^{4″})NR^{4′}-benzimidazolyl, —C(NR^{4″})NR^{4′}-benzthiazolyl, —C(NR^{4″})NR^{4′}-benzoxazolyl, or heteroaryl;
R^{4′}, R^{4″}, R^{5′} independently are H, halogen, alkyl, —C(NR⁷)NR^{7′}R⁸, —(CH₂)ₚaryl, haloalkyl, —(CH₂)ₚNR⁷R⁸, —C(O)NR⁷R⁸, —N═CR⁷R⁸, —NR⁷C(O)R⁸, cycloalkyl, heterocycloalkyl, hydroxyalkyl, hydroxyalkylamino, alkylamino, heteroaryl, or aryl.
p=1-6
q=1-6

4. A compound of formula (Ihb-4) or pharmaceutically acceptable salts thereof with an acid or a base, or a stereoisomer thereof, for the prevention and/or treatment of a disease associated with increased cytokine release

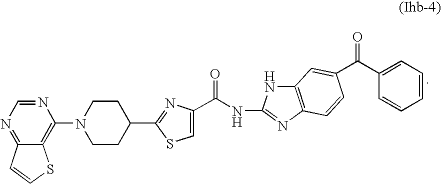

5. A pharmaceutical composition comprising a compound according to claim 1 and a pharmaceutically acceptable carrier or diluent, for the prevention and/or treatment of a disease associated with increased cytokine release.

6. A compound according to claim 1 for the treatment of a disease associated with increased cytokine release, whereby the disease is caused by a viral infection.

7. A compound according to claim 1 for the treatment of a disease associated with increased cytokine release, whereby the disease is caused by an infection with an influenza virus.

8. A compound according to claim 1 for the treatment of a disease associated with increased cytokine release, whereby the disease is caused by an infection with influenza virus of one or more of the subtypes H5N1, H2N2, H1N1 or H3N2.

9. A compound according to claim 1 for the treatment of a disease associated with increased cytokine release, whereby the disease is caused by a bacterial infection.

10. A compound according to claim 1 for the treatment of a disease associated with increased cytokine release, whereby the disease is an inflammatory disease.

11. A compound according to claim 1 for the treatment of a disease associated with increased cytokine release, whereby the disease is cancer.

12. A compound according to claim 1 for the treatment of a disease associated with increased cytokine release, whereby the disease is stroke or sepsis.

13. A compound according to claim 1 for the prevention and/or treatment of a disease associated with increased cytokine release, whereby the compound or composition is administered in combination with INFα.

14. A compound according to claim 1 for the prevention and/or treatment of an influenza virus infection associated with increased cytokine release, whereby the compound or composition is administered in combination with INFα.

15. A compound according to claim 1 for the prevention and/or treatment of a disease associated with an infection with one or more of the influenza virus subtypes H1N1, H5N1, H2N2 or H3N2, whereby the compound or composition is administered in combination with INFα.
